(19) United States
(12) Patent Application Publication
(10) Pub. No.: US 2022/0208374 A1
(43) Pub. Date: Jun. 30, 2022

(54) SYSTEMS AND METHODS FOR GENERATING AN INTEGRATIVE PROGRAM

(71) Applicant: KPN INNOVATIONS, LLC., LAKEWOOD, CO (US)

(72) Inventor: Kenneth Neumann, Lakewood, CO (US)

(73) Assignee: KPN INNOVATIONS, LLC., LAKEWOOD, CO (US)

(21) Appl. No.: 17/463,915

(22) Filed: Sep. 1, 2021

Related U.S. Application Data
(63) Continuation-in-part of application No. 17/136,102, filed on Dec. 29, 2020, now Pat. No. 11,164,669.

(51) Int. Cl. G16H 50/20; G06N 20/00

(52) U.S. Cl. CPC G16H 50/20; G06N 20/00

(57) ABSTRACT

A system for generating a viral alleviation program including a computing device configured to retrieve a viral epidemiological profile, identify, a integrative signature as a function of the viral epidemiological profile, wherein identifying further comprises receiving a behavioral indicator, and identifying the integrative signature as a function of the behavioral indicator and the viral epidemiological profile using a integrative machine-learning model, and generating a integrative program as a function of the integrative signature.

### CROSS-REFERENCE TO RELATED APPLICATIONS

This application is a continuation-in-part of Non-provisional application Ser. No. 17/136,102 filed on Dec. 29, 2020 and entitled “SYSTEMS AND METHODS FOR GENERATING A VIRAL ALLEVIATION PROGRAM,” the entirety of which is incorporated herein by reference.

### FIELD OF THE INVENTION

The present invention generally relates to the field of nutrition modeling for viral infection alleviation. In particular, the present invention is directed to generating an integrative program.

### BACKGROUND

It has been estimated that a large degree of viral infections may be preventable using a variety of strategies such as, masks, distancing, and handwashing. Although, priming the immunological response by pharmacological intervention, such as by vaccination, maintains the standard for prevention of viral infection.

### SUMMARY OF THE DISCLOSURE

In an aspect, a system for generating a viral alleviation program including a computing device configured to retrieve a viral epidemiological profile related to the user, identify, a integrative signature as a function of the viral epidemiological profile, wherein identifying further comprises receiving a behavioral indicator, and identifying the integrative signature as a function of the conduct indicate and the viral epidemiological profile using a integrative machine-learning model, and generate a integrative program as a function of the integrative signature.

In another aspect, a method for generating a viral alleviation program including retrieving, by the computing device, a viral epidemiological profile related to the user, identifying, by the computing device, a integrative signature as a function of the viral epidemiological profile, wherein identifying further comprises receiving a behavioral indicator, and identifying the integrative signature as a function of the conduct indicate and the viral epidemiological profile using a integrative machine-learning model, and generating, by the computing device, a integrative program as a function of the integrative signature.

These and other aspects and features of non-limiting embodiments of the present invention will become apparent to those skilled in the art upon review of the following description of specific non-limiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a block diagram illustrating a system for generating a viral alleviation program;

FIG. 2 is a block diagram illustrating a system for generating an integrative program;

FIG. 3 is a block diagram illustrating a machine-learning module;

FIG. 4 is a block diagram of an alleviation program database;

FIGS. 5A and 5B are a diagrammatic representation of a viral epidemiological profile;

FIG. 6 is a diagrammatic representation of a viral alleviation program;

FIG. 7 is a diagrammatic representation of a user device;

FIG. 8 is a block diagram of a workflow of a method for generating a viral alleviation program;

FIG. 9 is a block diagram of a workflow of a method for generating an integrative program; and

FIG. 10 is a block diagram of a computing system that can be used to implement any one or more of the methodologies disclosed herein and any one or more portions thereof.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to systems and methods for generating a viral alleviation program. In an embodiment, system includes a computing device configured to receive viral biomarkers of a user. Viral biomarkers may include experimental testing results, such as PCR test data, antigen testing, antibody testing, ELISA, etc. Computing device is configured to retrieve a viral epidemiological profile, which may include a variety of data used to generate a phylogenic profile of a virus. Computing device may generate viral epidemiological profile, by using a machine-learning algorithm to model viral incidence to a plurality of viral epidemiological factors, such as detection, transmission chain, and spread. Computing device is configured to determine the effect of nutrients on the user's viral epidemiological profile. Computing device may generate a spread model which models a plurality of effects of the plurality of nutrient amounts on viral spread rates, determining unique effects for a plurality of nutrients. Computing device may identify nutrition elements, such as individual ingredients, and calculate a viral alleviation program using the calculated nutrient amounts and their effects according to viral biomarkers. In an embodiment, computing device may accept user input and generate viral alleviation program, where elements are curated according to the user input. Participation and adherence to viral alleviation program may be used to provide a viral alleviation metric. Computing device may calculate changes in the incidence of viral infection in the user according to adherence to viral alleviation program.

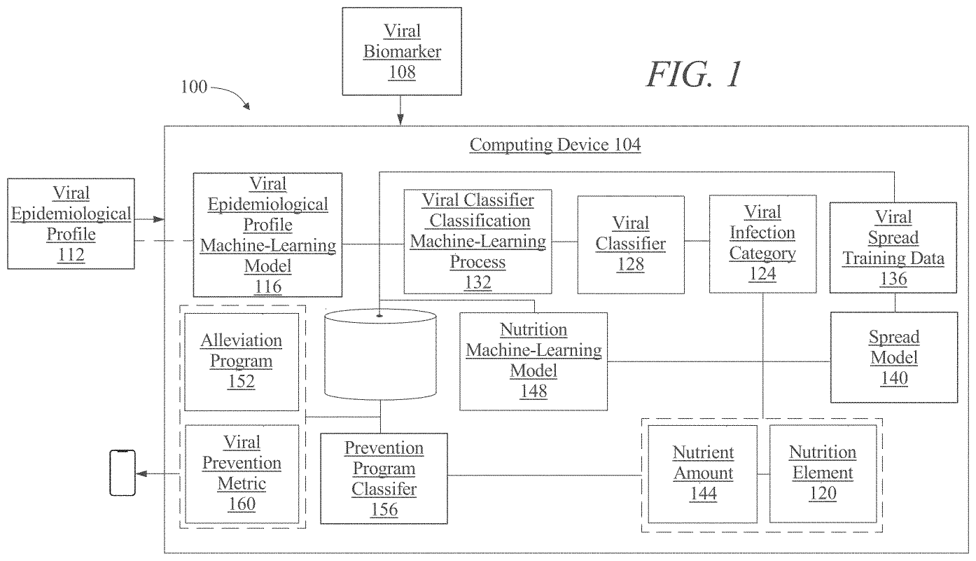

Referring now to FIG. 1, an exemplary embodiment of a system 100 for generating a viral alleviation program is illustrated. System includes a computing device 104. Computing device 104 may include any computing device as described in this disclosure, including without limitation a microcontroller, microprocessor, digital signal processor (DSP) and/or system on a chip (SoC) as described in this disclosure. Computing device may include, be included in, and/or communicate with a mobile device such as a mobile telephone or smartphone. Computing device 104 may include a single computing device operating independently, or may include two or more computing device operating in concert, in parallel, sequentially or the like; two or more computing devices may be included together in a single computing device or in two or more computing devices. Computing device 104 may interface or communicate with one or more additional devices as described below in further detail via a network interface device. Network interface device may be utilized for connecting computing device 104 to one or more of a variety of networks, and one or more devices. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network may employ a wired and/or a wireless mode of communication. In general, any network topology may be used. Information (e.g., data, software etc.) may be communicated to and/or from a computer and/or a computing device. Computing device 104 may include but is not limited to, for example, a computing device or cluster of computing devices in a first location and a second computing device or cluster of computing devices in a second location. Computing device 104 may include one or more computing devices dedicated to data storage, security, distribution of traffic for load balancing, and the like. Computing device 104 may distribute one or more computing tasks as described below across a plurality of computing devices of computing device, which may operate in parallel, in series, redundantly, or in any other manner used for distribution of tasks or memory between computing devices. Computing device 104 may be implemented using a “shared nothing” architecture in which data is cached at the worker, in an embodiment, this may enable scalability of system 100 and/or computing device.

With continued reference to FIG. 1, computing device 104 may be designed and/or configured to perform any method, method step, or sequence of method steps in any embodiment described in this disclosure, in any order and with any degree of repetition. For instance, computing device 104 may be configured to perform a single step or sequence repeatedly until a desired or commanded outcome is achieved; repetition of a step or a sequence of steps may be performed iteratively and/or recursively using outputs of previous repetitions as inputs to subsequent repetitions, aggregating inputs and/or outputs of repetitions to produce an aggregate result, reduction or decrement of one or more variables such as global variables, and/or division of a larger processing task into a set of iteratively addressed smaller processing tasks. Computing device 104 may perform any step or sequence of steps as described in this disclosure in parallel, such as simultaneously and/or substantially simultaneously performing a step two or more times using two or more parallel threads, processor cores, or the like; division of tasks between parallel threads and/or processes may be performed according to any protocol suitable for division of tasks between iterations. Persons skilled in the art, upon reviewing the entirety of this disclosure, will be aware of various ways in which steps, sequences of steps, processing tasks, and/or data may be subdivided, shared, or otherwise dealt with using iteration, recursion, and/or parallel processing.

Continuing in reference to FIG. 1, computing device is configured to receive at least a viral biomarker related to a user. A “viral biomarker,” as used in this disclosure, is a biological and/or chemical substance or process that is indicative of the presence of viral infection in the body. Viral biomarker 108 may include biological molecules existing within a normal cell, an infected cell, secreted by a viral-infected cell, and/or a specific response of the body to the presence of viruses. Receiving at least the viral biomarker 108 may include receiving a result of one or more tests relating the user. Viral biomarker 108 may include test results of screening and/or early detection of viruses, for instance from a PCR test (qPCR, qtPCR, RTPCR etc.), viral antigen test, antibody test, enzyme-linked immunosorbent assay (ELISA), T-cell activation test, among other biochemical data. Test may include diagnostic procedures, prognostic indicators from other diagnoses, from predictors identified in a medical history, and information relating to biomolecules associated with viral infection such as: interleukin (IL) IL-1B, IL-1-RA, IL-2, IL-6, IL-8, IL-10, MIP-1alpha, MIP-1Beta, MCP-1, MCSF, MIF, IP-10, GRO-alpha, eotaxin, neopterin, sTNF-RI, TNF-alpha, sFasL, sFAS, IFN-alpha, IFN-gamma, CCL17, CXCL5, CXCL9, IP-10, CXCL11, mixed lineage kinase domain-like protein (MLKL), urea, creatinine, cystatin C, bilirubin, cholinesterase, procalcitonin, and the like. Persons skilled in the art may appreciate the full spectrum of biochemical data relating to the body that may be indicative of viral infection and/or may constitute a “viral biomarker,” as described herein.

Continuing in reference to FIG. 1, test may include results enumerating the relationships between proteins, DNA, RNAs, white blood cells, among other macromolecules, signaling peptides, and the like, such as increases/decreases in concentrations of cytokines, ratios of cytokine concentrations, complement pathway proteins, phosphorylation states, presence of dsRNA, and the like. Test results may indicate the presents of genetic insertions, deletions, translocations, inversions, gene expression levels, single nucleotide polymorphisms (SNPs), etc., in genetic sequences that may make an individual more or less susceptible to a particular viral infection. For instance and without limitation, among Caucasians, a 32-base-pair deletion in the coding region of the chemokine receptor, CCR-5, which renders such individuals far more resistant to HIV-1 than those with intact alleles. Test results may indicate blood panel factors, microbiological factors, epigenetic factors, among other categories of biological, physiological, and chemical indicators of viral infection. Test may include a health state questionnaire, where a user may indicate a symptom relating to viral infection. Viral biomarker 108 may include hematological analysis including results from T-cell activation assays, abnormal nucleation of white blood cells, white blood cell counts, concentrations, recruitment, localization, and the like. Viral biomarker 108 may be received as a function of a user indicating a prior diagnosis, or a current medication, wherein one is a viral infection treatment, such as for treatment of Herpes, Hepatitis, HIV, Epstein-Barr virus, among other chronic and acute viral pathologies. Viral biomarker 108 may include any virus-related symptoms, side effects, and co-morbidities associated with and relating to viral infection diagnosis. Viral biomarker 108 may be received and/or identified from a biological extraction of a user, which may include analysis of a physical sample of a user such as blood, DNA, saliva, stool, and the like, without limitation and as described in U.S. Nonprovisional application, Ser. No. 16/886,647, filed May 28, 2020, and entitled, “METHODS AND SYSTEMS FOR DETERMINING A PLURALITY OF BIOLOGICAL OUTCOMES USING A PLURALITY OF DIMENSIONS OF BIOLOGICAL EXTRACTION USER DATA AND ARTIFICIAL INTELLIGENCE,” the entirety of which is incorporated herein by reference.

Continuing in reference to FIG. 1, viral biomarker 108 may be organized into training data sets. “Training data,” as used herein, is data containing correlations that a machine learning process, algorithm, and/or method may use to model relationships between two or more categories of data elements. For instance, and without limitation, training data may include a plurality of data entries, each entry representing a set of data elements that were recorded, received, and/or generated together; data elements may be correlated by shared existence in a given data entry, by proximity in a given data entry, or the like. Multiple data entries in training data may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data according to various correlations; correlations may indicate causative and/or predictive links between categories of data elements, which may be modeled as relationships such as mathematical relationships by machine learning processes as described in further detail below.

Continuing in reference to FIG. 1, viral biomarker 108 may be used to generate training data for a machine-learning process. A “machine learning process,” as used in this disclosure, is a process that automatedly uses a body of data known as “training data” and/or a “training set” to generate an algorithm (such as a collection of one or more functions, equations, and the like) that will be performed by a machine-learning module to produce outputs given data provided as inputs; this is in contrast to a non-machine learning software programing where the commands to be executed are determined in advance by a subject and written in a programming language, as described in further detail below.

Continuing in reference to FIG. 1, viral biomarker 108 may be organized into training data sets and stored and/or retrieved by computing device 104, without limitation, as a relational database, a key-value retrieval database such as a NOSQL database, or any other format or structure for use as a database that a person skilled in the art would recognize as suitable upon review of the entirety of this disclosure. Viral biomarker 108 training data may alternatively or additionally be implemented using a distributed data storage protocol and/or data structure, such as a distributed hash table and the like. Viral biomarker 108 training data may include a plurality of data entries and/or records, as described above. Data entries may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries of viral biomarkers may be stored, retrieved, organized, and/or reflect data and/or records as used herein, as well as categories and/or populations of data consistent with this disclosure.

Continuing in reference to FIG. 1, computing device is configured to retrieve a viral epidemiological profile related to the user. A “viral epidemiological profile,” as used in this disclosure, is a profile that summarizes a user's current state of viral infection in themselves and the epidemiology of viruses in the community about the user. A “community,” as used in this disclosure, is a particular region of interest for viral epidemiology; a community may include varying degrees of granularity depending on the region selected (town, city, county, state, country, etc.).Viral epidemiological profile 112 may include at least an epidemiological factor, A “epidemiological factor,” as used in this disclosure, is a quantitative metric that encapsulates epidemiology of a virus-user relationship. Viral epidemiological profile 112 may include qualitative and/or quantitative parameters which capture the presence of clinical manifestation of viral infection in the user, as well as the detection of and incidence of viral infection in the community. Viral epidemiological profile 112 may include qualitative determinations, such as binary “yes”/“no” determinations for viral infection types, “normal”/“abnormal” determinations about the presence of and/or concentration of viral biomarkers 108, for instance as compared to a normalized threshold value of a viral biomarker 108 among healthy adults. Viral epidemiological profile 112 may include a plurality of epidemiological factors, wherein epidemiological factors are quantitative determinations such as a “viral infection score”, which may include any metric, parameter, or numerical value that communicates a viral state. Viral epidemiological profile 112 may include epidemiological factors that are mathematical representations of the current state of viral infection, such as a function describing the viral infection risk as a function of time (with age, changing diet, sleep patterns, time of year, climate, etc.). Epidemiological factors may be virus-specific, climate-specific, tissue-specific, biological pathway-specific, etc. Viral epidemiological profile 112 may include instantaneous viral infection risk, such as weekly, monthly, annual, etc., classified by virus/infection type, according to medical history, biological extraction test result, and the like.

Continuing in reference to FIG. 1, viral epidemiological profile 112 may include a plurality of epidemiological factors involving viral epidemiology for a plurality of viruses, including: 1) detection of virus, 2) targeting of virus, 3) transmission chain, and 4) spread, among other factors. Detection of virus may include tracking of case numbers by testing such as PCR, ELISA, antibody, etc., and/or metagenomic sequencing methods which matches a disease to a viral identity. Targeting of virus may include targeted sequencing from additional human cases and from related viruses uncovering the likely animal reservoir, the time period that it was introduced into the human population, and confirmation about subsequent transmission (e.g. human-to-human, animal-to-human, etc.). Transmission chain may include more intensive virus genome sequencing used to construct detailed transmission chains and identify potential control measures. Spread may include layering additional climatic, transportation, geographic, economic, and demographic data into a large phylogenetic dataset revealing the risk factors that facilitate local and global spread and how this may relate to a user's viral infection risks. This may include training a machine-learning model to build a phylogenetic model which has viral incidence mapped as a function of strain-disease relationships. Spread may use sampling of data, for instance from the Internet, to determine if a viral outbreak is imminent, occurring, etc. Viral epidemiological profile 112 may include modeling disease spread to symptom and to diet to reverse symptomology, improve immunity, and prevent viral infection to build a viral alleviation program, as described in further detail below.

Continuing in reference to FIG. 1, retrieving viral epidemiological profile 112 may include receiving viral epidemiological profile training data. “Viral epidemiological profile training data,” as used in this disclosure, is training data sets used for training a machine-learning process, algorithm, and/or model, for the purpose of deriving a viral epidemiological profile of a user. Viral epidemiological profile training data may include viral biomarkers 112 organized into training data sets, as described above. Viral epidemiological profile training data may originate from the user input, for instance via an interaction with computing device 104, to provide medical history data. Viral epidemiological profile training data may originate from a set of users, for instance test result data among the city, state, county, etc., from Center for Disease Control (CDC), municipal health departments, and the like. Viral epidemiological profile training data may include receiving whole genome sequencing, gene expression patterns, and the like, for instance as provided by a genomic sequencing entity, hospital, researchers, database, etc. Viral epidemiological profile training data may be recorded and transmitted to computing device 104 via a wearable device such as a pedometer, gyrometer, accelerometer, motion tracking device, bioimpedance device, ECG/EKG/EEG monitor, physiological sensors, blood pressure monitor, blood sugar and volatile organic compound (VOC) monitor, and the like, which may help transmit user symptomology prior to user realization, such as increases and/or decreases in blood pressure, resting heart rate, oxygenation, etc. Training data may originate from an individual other than user, including for instance a physician, lab technician, nurse, epidemiologist, researcher, and the like. Viral epidemiological profile training data may include a plurality of data entries of viral biomarkers for instance as categorized by user cohort—i.e. HPV-positive users, Hepatitis C-positive users, HIV-positive users, users in high viral incidence, low viral incidence, overweight users, users with high fitness levels, immunocompromised users, etc. Such training data may be used to compare user to more accurately define parameters in viral epidemiological profile 112 relative to the overall population.

Continuing in reference to FIG. 1, in non-limiting illustrative examples, training data as described herein, may be input into computing device 104 for instance via a health state questionnaire for onboarding of user symptomology, via any graphical user interface. A “graphical user interface,” as used in this disclosure, is any form of a user interface that allows a user to interface with an electronic device through graphical icons, audio indicators, text-based interface, typed command labels, text navigation, and the like, wherein the interface is configured to provide information to the user and accept input from the user. Graphical user interface may accept input, wherein input may include an interaction (such as a questionnaire, embedding a hyperlink, uploading a document, etc.) with a user device. A user device, as described in further detail below, may include computing device 104, a “smartphone,” cellular mobile phone, desktop computer, laptop, tablet computer, internet-of-things (IOT) device, wearable device, among other devices. User device may include any device that is capable for communicating with computing device 104, database, or able to receive data, retrieve data, store data, and/or transmit data, for instance via a data network technology such as 3G, 4G/LTE, 5G, Wi-Fi (IEEE 802.11 family standards), and the like. User device may include devices that communicate using other mobile communication technologies, or any combination thereof, for short-range wireless communication (for instance, using Bluetooth and/or Bluetooth LE standards, AirDrop, Wi-Fi, NFC, etc.), and the like.

Continuing in reference to FIG. 1, retrieving viral epidemiological profile 112 may include training a viral epidemiological profile machine-learning model with training data that includes a plurality of data entries wherein each entry correlates viral biomarkers 108 to a plurality of epidemiological factors. Viral epidemiological profile machine-learning model 116 may include any machine-learning algorithm (such as K-nearest neighbors algorithm, lazy naïve Bayes algorithm, etc.), machine-learning process (such as supervised machine-learning, unsupervised machine-learning), or method (such as neural nets, deep learning, etc.). Viral epidemiological profile machine-learning model 116 may be trained to derive the algorithm, function, series of equations, or any mathematical operation, relationship, or heuristic, that may be generated to automatedly accept an input (viral biomarker(s) 108) and correlate, classify, or otherwise calculate an output (viral epidemiological profile 112). Viral epidemiological profile machine-learning model 116 may include individual functions, derived for unique relationships observed from the training data, for instance for each viral biomarker 108. In non-limiting illustrative examples, the expression levels of a variety of cytokines, as identified above, may be retrieved from a database, such as a repository of peer-reviewed research (e.g. National Center for Biotechnology Information is part of the United States National Library of Medicine), and the viral epidemiological profile machine-learning model 116 derived algorithm may observe an average and statistical evaluation (mean±S.D.). And, this may be calculated from the data, across which the user's expression level is compared. In such an example, viral epidemiological profile machine-learning model 116 may derive an algorithm according to the data which may also include a scoring function that demonstrates a relationship for how to arrive at a numerical value score according to the user's level of gene expression (e.g. mRNA transcripts of a cytokine per tissue, the cytokine level in blood, etc.) as it relates to the average and statistical evaluation in normal expression of cytokines, such as interleukins.

Continuing in reference to FIG. 1, viral epidemiological profile 112 may include data regarding a plurality of epidemiological factors. Plurality of epidemiological factors may include the factors, as described above. Epidemiological factors may also include, for instance and without limitation, what virus is causing an outbreak: metagenomic sequencing may be used from patient samples to reveal a novel virus—such as Lujo virus—as the causal virus for an outbreak such as in South Africa in 2008; how is the virus transmitting: for instance, sequencing studies of MERS coronavirus combined with coalescent approaches may show that human outbreaks are driven by seasonally varying zoonotic transfer of viruses from camels; where did the outbreak begin: large-scale sequencing efforts and phylogenetic analyses may show, for instance, that the 2009 influenza A/H1N1 pandemic originated in swine populations from Mexico; what factors drive the outbreak: analysis, for example, of more than 1,600 Ebola virus genomes identified critical factors that contributed to the spread of the virus during the 2013-2016 epidemic in West Africa; how many introductions have there been: sequencing data, for instance, of Zika virus from patients and mosquitos in Florida may show that multiple introduction events of the virus sustained the 2016 outbreak in Miami and surrounding counties; when did the outbreak begin: large-scale metagenomic studies, for instance, may show that the Zika epidemic in the Americas likely started in Brazil more than a year earlier than was initially believed; are outbreaks linked: analysis, for instance, of Ebola virus genomes during the 2013-2016 epidemic may show that the virus may persist for more than a year in survivors, and be responsible for flare-ups of the outbreak via sexual transmission; how is the virus evolving: sequencing studies, for example, during the 2013-2016 Ebola epidemic identified mutations in the virus genome that rapidly rose to a high frequency, compatible with increased fitness; experimental follow-up studies showed that some of those mutations were probably Ebola virus adapting to a new host. Such data, when summarized in viral epidemiological profile 112, may be used to derive novel equations to calculate nutrient amounts, relate nutrient amounts to foods, and time the consumption of the food to increase preparedness in handling viral infection, as described in further detail below more accurately.

Continuing in reference to FIG. 1, viral biomarker 108 may be correlated to a plurality of epidemiological factors without the use of machine-learning process(es). For instance and without limitation, computing device 104 may use a web browser and the Internet to identify a plurality of threshold values of cytokine levels that relate to viral biomarkers 108 in “healthy adults”, wherein gene expression values that deviate from such a threshold may indicate a particular viral infection, and the magnitude of deviation relates to the magnitude of a numerical value associated therewith.

Continuing in reference to FIG. 1, retrieving the viral epidemiological profile 112 may include generating the viral epidemiological profile 112 using the viral epidemiological profile machine-learning model 116 and at least a viral biomarker 108. Persons skilled in the art may appreciate that viral epidemiological profile 112 may become increasingly more complete, and more robust, with training data describing larger sets of viral biomarkers 108 in the user, training data describing infection incidence with higher granularity, metagenomic data highlighting strain-disease relationships, etc. Epidemiological factors may be generated for each viral biomarker 108 (or set of biomarkers) as described above; epidemiological factors may be generated for each individual virus (e.g. Rhinovirus), viral strain (e.g. Rhinovirus A 100-103), and/or viral type (e.g. cold viruses). Epidemiological factors may be generated for each time of year, climate, weather, etc. Epidemiological factors may be generated for each white blood cell type (e.g. Neutrophils, Macrophages, Basophils, etc.), or set of white blood cell type, among other factors, where user immunological function is related to risk, incidence, and spread.

Continuing in reference to FIG. 1, viral epidemiological profile machine-learning model 116 may derive a unique equation, algorithm, and/or set of functions for determining individual epidemiological factors. Viral epidemiological profile machine-learning model 116 may derive functions, systems of equations, matrices, variables, etc., that describe and/or incorporate relationships between sets of viral biomarkers 108 (training data), for instance and without limitation, combining the expression level of two or more cytokines, multiplied by scalar coefficients according to the presence of SNPs (single nucleotide polymorphisms) or mutations present in the viral host receptors, dividing by the ratio of phosphorylated-unphosphorylated states, ubiquitinated states, etc., of the cytokines. In such an example, said data may determine if a user is currently experiencing a viral infection, and of which particular infection category. In the full spectrum of cell signaling, maintaining cellular homeostasis, cell division, protein degradation, among other biological phenomenon that may contribute to the progression of viral infection, viral epidemiological profile machine-learning model 116 may derive increasingly complicated algorithms for combining viral biomarkers 108 into epidemiology factors summarized in viral epidemiological profile 112.

Continuing in reference to FIG. 1, computing device 104 is configured to identify, using the viral epidemiological profile 112, a plurality of nutrition elements for the user. A “nutrition element,” as used in this disclosure, is an item that includes a nutrient intended to be used and/or consumed by user for treating and/or preventing viral infection. A “nutrient,” as used in this disclosure,” is a biologically active compound whose consumption is intended for the treatment and/or prevention of viral infection. Nutrition element 120 may include alimentary elements, such as meals (e.g. chicken parmesan with Greek salad and iced tea), food items (e.g. French fries), grocery items (e.g. broccoli), health supplements (e.g. whey protein), beverages (e.g. orange juice), and the like. Nutrition element 120 may be “personalized” in that nutrition elements are curated in a guided manner according to viral epidemiological profile 112, gene expression patterns, viral biomarkers 108, SNPs, the time of year, a viral category (e.g. respiratory infection, common cold, tissue tropism, etc.), and the like. Nutrition element 120 may include supplementary use of oral viral epidemiologic enzymes and probiotics which may also have merit as antiviral measures. Nutrition elements 120 in a virus prevention diet may include zinc, selenium, vitamin A, vitamin C, vitamin D, vitamin E, cyanocobalamin, N-acetyl-cysteine (NAC), and antioxidants such as the carotenoids (α-carotene, β-carotene, lycopene, lutein, cryptoxanthin). Nutrient elements 120 may contain biological active compounds that are not typically considered vitamins and/or minerals, nor are they intended to provide appreciable amounts of calories, such as phytonutrients and nutraceuticals; for instance allium and bioactive ingredients present in cruciferous vegetables such as broccoli sprouts, a known source of antioxidants such as sulforaphane, which may have therapeutic effects on viral infection. Nutrition elements 120 may include a specific dietary category, such as a “ketogenic diet”, “low glycemic index diet”, “Paleo diet”, and so on.

Continuing in reference to FIG. 1, identifying the plurality of nutrition elements 120 includes assigning the viral epidemiological profile 112 to a viral infection category, wherein the viral infection category is a determination about a current viral epidemiological state of the user. A “viral infection category,” as used in this disclosure, is a designation of the current viral epidemiological state of a user and/or the community of the user according to data in the viral epidemiological profile. Viral infection category 124 may differ from viral epidemiological profile 112 in that the viral infection category 124 may include a determination about a user's viral state according to how they may be classified as a function of a subset of user cohort. In non-limiting illustrative examples, viral epidemiological profile 112 may include data indicating a current symptomology of ‘dry cough’, ‘shortness of breath’, and ‘fever’, along with a current incidence rate associated with SARS-Cov-2 virus in the user's county; however, the user may not be diagnosed with COVID-19, in which case the user may be classified to such a viral infection category 124 according to the patterns in symptomology and incidence rate data in the viral epidemiological profile 112 of the user. This may be performed for any number of viral categories, in which the user may be preemptively categorized to a disease category based on the propensity of infection according to the data in the viral epidemiological profile 112. Viral infection category 124 may include any medical, physiological, biological, chemical, and/or physical determination about the current state of a user's propensity for viral infection, including their “current status for viral infection” (HIV, HSV, HPV, Hep A/B/C status, etc.), and projected, future likelihood for viral infection, wherein current viral infection and future likelihood are linked by incidence rates, such as instantaneous rate, future rate, etc. The viral epidemiological profile 112 may include data that summarizes the user's biomarkers, symptomology, rates of infection in the community; whereas the viral infection category 124 is a determination about a probable diagnosis or future diagnosis based on patterns observed in symptoms, biomarkers, rates of infection, and the like, from cohorts of users.

Continuing in reference to FIG. 1, viral infection category 124 may include tissue or organ type, such as “liver virus”, “lung virus”, etc. Viral infection category 124 may include generic body system classification such as encephalitis, meningitis, Common cold, eye infections, pharyngitis, gingivostomatitis, parotitis, pneumonia, cardiovascular, hepatitis, pancreatitis, myelitis, skin infections, sexually transmitted disease, gastrointestinal, etc. Viral infection category 124 may include a designation regarding a viral category that may not involve a particular tissue such as “Respiratory infection”, “Diarrheal Virus”, etc. Viral infection category 124 may include designations about a group of viral serotypes such as Coxsackie B virus, a clade of virus such as alpha/beta/gamma Coronavirus, viral strain such H1N1 Influenza A, viral incidence rates (e.g. high daily likelihood of infection, low daily likelihood, etc.), transmission chain (e.g. human-to-human rate, reservoir identity, reservoir-to-human rate, etc.), among other data that may be determined on a per-viral serotype, clade, strain, etc. basis. Viral infection category 124 may include identifiers associated with severity of infection (mortality and/or morbidity), infection rates (rate of infection, IC50, LD50, titers, IFUs/mL, PFU/mL, etc.), survivability (percentiles, etc.), among other data that may be determined on a per-viral infection basis. Viral infection category 124 may include a predictive viral infection classification, where a user does not currently harbor a particular viral infection but may include data that indicates a viral infection category 124 with which they may be most closely categorized to, imminently infected, etc. For instance, a user who has not received a flu shot, who lives in a temperate climate in the Fall season of the Northern Hemisphere, in a community where cold and flu infections have significantly risen (as summarized in the user's viral epidemiological profile 112) may classify an individual to “Common Cold” or “Respiratory Infection” viral infection category 124, despite not currently exhibiting either. Viral epidemiological profile 112 may have associated with it an identifier, such as a label, that corresponds to a viral infection category 124.

Continuing in reference to FIG. 1, assigning the viral epidemiological profile to a viral infection category may include training a viral classifier using a viral classification machine-learning process and training data which includes a plurality of data entries wherein each data entry correlates viral biomarkers 108 to a viral infection category 124. A “viral classifier,” as used in this disclosure, is a machine-learning model as defined herein, such as a mathematical model, neural net, or program generated by a machine learning algorithm known as a “classification algorithm,” as described in further detail below, that sorts inputs into categories or bins of data, outputting viral infection category 124, or bins of data and/or labels associated therewith.

Continuing in reference to FIG. 1, training viral classifier 128 to classify user to viral infection category 124 may include using a viral classification machine-learning process. Classification may include identifying which set of categories (viral infection category 124) an observation (viral biomarker 108) belongs. Alternatively or additionally, observations associated with epidemiological profile 112 may be classified to viral infection category 124. Viral classifier 128 may include classification based on clustering as a function of pattern recognition, wherein the presence of certain viral biomarkers 108 and/or viral epidemiological factors, such as genetic indicators, symptoms, community incidence, and the like, relate to a particular viral infection category 124. Such classification methods may include binary classification (with or without the use of machine-learning), where the viral epidemiological profile 112 is matched to each existing viral infection category 124 and sorted into a category based on a “yes”/“no” match. Classification done in such a manner may include weighting, scoring, or otherwise assigning a numerical value to elements in viral epidemiological profile 112 as it relates to each viral category and assign a user to a viral infection category 124 for the viral type that results in the highest ‘score’. Such a ‘score’ may represent a “likelihood”, probability, or other numerical data that relates to the classification into viral infection category 124.

Continuing in reference to FIG. 1, viral classification machine-learning process may include any machine-learning process, method, and/or algorithm, as described in further detail below. Viral classification machine-learning process 132 may generate a viral classifier 128 using training data. Viral classifier 128 may be configured to output at least a datum that labels or otherwise identifies a set of data that are clustered together, found to be close under a viral infection category 124, among other classification, as described herein. Machine-learning module, as described in further detail below, may generate viral classifier 128 using a classification algorithm, defined as a process whereby a computing device and/or any module and/or component operating thereon derives a classifier from training data. As a non-limiting example, viral epidemiological profile 112 may be used as training data for viral classifier 128 which may be trained to classify elements of training data to elements that characterizes a sub-population, such as a subset of viral biomarker 108 (such as gene expression patterns as it relates to a variety of viral infection types) and/or other analyzed items and/or phenomena for which a subset of training data may be selected.

Continuing in reference to FIG. 1, assigning the viral epidemiology profile 112 to a viral infection category 124 may include assigning the viral infection category 124 as a function of the viral classifier 128. Viral classifier 128 may classify viral biomarker 108 (input) to viral infection category 124 (output) may include assigning the viral infection category 124 as a function of the viral classification machine-learning process 132 and the viral epidemiological profile 112 (e.g. summary of viral biomarker(s) 108, among data originating from outside the user). Training data may include sets of epidemiological factors and/or viral biomarkers 108, as described above. Such training data may be used to “learn” how to categorize a user's viral epidemiological profile 112 to viral infection categories 124 depending on trends in viral biomarkers 108, gene expression, SNPs, user symptomology, rate of incidence, and the like. Such training data may be used to anticipate a type of viral infection (viral infection category 124) from viral epidemiology profile 112 as a viral outbreak threat prior to the outbreak occurring. Training data for such a classifier may originate from user input, for instance via a health state questionnaire via a graphical user interface, may originate from a biological extraction test result such as genetic sequencing, blood panel, lipid panel, etc. Training data may originate from a user's medical history, a wearable device, a family history of disease. Training data may similarly originate from any source, as described above, for viral biomarker 108 and determining viral epidemiological profile 112.

Continuing in reference to FIG. 1, identifying the plurality of nutrition elements 120 includes calculating, according to the viral infection category 124, a plurality of nutrient amounts, wherein calculating a plurality of nutrient amounts includes determining an effect of the plurality of nutrient amounts on the viral epidemiological profile 112. An “effect of a nutrient,” as used in this disclosure, is a change, consequence, and/or result in at least a viral biomarker 108, viral epidemiological profile 112, viral infection category 124, and/or likelihood of viral infection in a user due to consumption of an amount of a nutrient. An effect of a nutrient may be “no effect”. An effect of a nutrient may include a resultant increase/decrease in viral biomarker 108. An effect of a nutrient may include in increase/decrease in likelihood of viral infection. An effect of a nutrient may include a change in viral infection category 124. Calculating an effect of a nutrient may include determining how a viral biomarker 108 may change, such as an increase/decrease according to a particular amount of nutrient. For instance and without limitation, such a calculation may include determining the effect on cytokine levels from chronic, sustained nutrient amounts in a diet for weeks, months, etc.

Continuing in reference to FIG. 1, determining the effect of the plurality of nutrient amounts on the viral epidemiological profile 112 may include receiving viral spread training data. “Viral spread training data,” as used in this disclosure, is data for training a machine-learning process, algorithm, and/or model which includes a plurality of data entries that correlates a plurality of nutrient amounts to viral spread rates in a region. Viral spread training data 136 may be used to determine “idealized nutrient amounts,” wherein a machine-learning process may train with such data to derive nutrient amounts, that if consumed by the population at large, may reduce overall spread of a virus through that population. Viral spread training data 136 may include any type of training data described herein, for instance viral biomarkers 108 for a user, subset of users, incidence of a virus in a community over time, etc. Viral spread training data 136 may include any data concerning the epidemiological factors, such as data of positive cases of viral infection per number of individuals tested originating from municipal health departments, clinics, Centers for Disease Control (CDC), hospitals, and the like, as described herein. Viral spread training data 136 may include data concerning the use of public transit systems. In non-limiting illustrative examples, such training data may include the number of individuals who use the subway, buses, aircraft, etc., in a region related to the number of positive cases of a viral outbreak in the region. Such training data may be used to derive relationships from the data that corresponds to spread of a virus, wherein each type of transit has a particular risk factor.

Continuing in reference to FIG. 1, determining the effect of the plurality of nutrient amounts on the viral epidemiological profile 112 may include a hypothesis (model) about the user's health after consuming a nutrient amount. An effect of a plurality of nutrient amounts may include the effect on viral infection category 124 (e.g. changing from “Common Cold” to “Respiratory Virus”), viral biomarker 108 (e.g. decreasing amounts of cytokines, etc.), likelihood of viral infection (e.g. 60% to 20% chance), risk (very high, high, medium, etc.), and the like, from a particular nutrient amount, or combination of nutrient amounts. In non-limiting illustrative examples, determining an effect of a nutrient may include determining if a change in viral infection category 124 may arise from adding and/or removing a nutrient from a user's diet, for instance changing a viral infection category 124 from “Common Cold” to “Respiratory Virus,” as increasing dietary vitamin C, zinc, and vitamin D may reduce risk from Rhinoviruses, Parainfluenza viruses, RSV, etc., but due to weather, climate, and incidence, user remains in “Respiratory Virus” category due to Influenza, Adenovirus, Coronavirus.

Continuing in reference to FIG. 1, determining the effect of the plurality of nutrient amounts on the viral epidemiological profile 112 may include generating a spread model, wherein the spread model is a machine-learning model trained with the viral spread training data 136 which includes a plurality of data entries that models a plurality of effects of the plurality of nutrient amounts on viral spread rates. A “spread model,” as used in this disclosure, is a machine-learning model generated from viral spread training data 136, which includes the phylogenetics and epidemiology of a virus in a human population. Spread model 140 may include “idealized” viral epidemiology modeling, wherein the rates of spread of a viral outbreak are tested against varying nutrient consumption in the population. In non-limiting illustrative examples, spread model 140 may derive a series of function that calculates how an individual's risk of infection changes if the population met a particular nutrition amount threshold. In such an example, nutrient amounts at local minima of the functions may represent the most reduced viral risk for those nutrients.

Continuing in reference to FIG. 1, determining the effect of the plurality of nutrient amounts on the viral epidemiological profile 112 may include determining the effect of the plurality of nutrient amounts as a function of the spread model 140. Spread model 140 may be increasingly robust, in that it may include retrieving and incorporating overlaid data structures relating public transportation, weather and climate, rates of incidence of a particular virus, such as COVID-19 (SARS-CoV-2 viral strain responsible for 2020 pandemic), among other types of data, to generate a model of the spread of the virus in a community. Such a spread model 140 may be used to inform nutrient amounts and timing of nutrient consumption due to the effect of nutrients on the spread of a virus (population phylogenetics—i.e. if x nutrient is consumed among population, spread is reduced by y amount). Alternatively, spread model 140 may be used to optimally time nutrient consumption by an amount—e.g. within 24 hrs. of case spike, following week of temperature below 60 degrees Fahrenheit, etc., to best prevent infection. Spread model 140 output may include mapping a series of points, such as numerical values per county in the United States, overlaid over a regional map. Each county may have a value assigned associated with the active number of cases of a viral outbreak, weather conditions conducive to viral spread (e.g. temp, humidity), population density, etc. Spread model 140 may incorporate these values, for instance as variables in a function, wherein spread model 140 may solve for a series of values, or singular value, which may be presented geographical using a mapping application, such as a heat map over a 500 mile radius. In such an instance, spread model 140 may be trained with data as each day passes to accurately derive how each variable affects the number of cases in each succeeding day.

Continuing in reference to FIG. 1, computing device 104 is configured for calculating the plurality of nutrient amounts as a function of the effect, wherein the plurality of nutrient amounts is a plurality of amounts intended to result in prevention of a viral infection. A “nutrient amount,” as used in this disclosure, is a numerical value(s) relating to the amount of a nutrient. Nutrient amount 144 may include mass amounts of a vitamin, mineral, macronutrient (e.g. grams carbohydrate, protein, fat), a value of calories, mass amounts, units, and/or concentrations of phytonutrients, antioxidants, pharmaceuticals, bioactive ingredients, and the like.

Continuing in reference to FIG. 1, calculating the plurality of nutrient amounts may include determining the plurality of nutrient amounts as a function of at least a viral biomarker 108 and the plurality of effects. Determining a nutrient amount may include a mathematical operation, such as subtraction, addition, etc. For instance and without limitation, spread model 140 may be used to derive an effect wherein a +5 mg daily nutrient amount of a vitamin combination results in a 10% decreased risk of viral infection, up to +40% benefit; in such an instance, a maximal benefit may be found, and nutrient amount calculated. Determining a nutrient amount may include retrieving an empirical equation that describes relationships between a nutrient and viral biomarker 108, test results, climate, and viral spread, etc. Determining a nutrient amount may include deriving an algorithm, function, or the like, to generate a plurality of nutrient amounts more accurately as a function of the viral epidemiological profile 112.

Continuing in reference to FIG. 1, computing device 104 may calculate nutrient amounts 144, for instance, by retrieving a default amount, such as from a standard 2,000 calorie diet, and modifying the amount according to viral epidemiological profile 112. Such a calculation may include a mathematical operation such as subtraction, addition, multiplication, etc.; alternatively or additionally, such a calculation may involve deriving a loss function, vector analysis, linear algebra, system of questions, etc., depending on the granularity of the process. Deriving such a process for calculating nutrient amounts 144 may include machine-learning. Nutrient amounts 144 may include threshold values, or ranges of values, for instance and without limitation, between 80-120 mg vitamin C per 24 hours, wherein the minimal acceptable blood concentration of ascorbic acid must stay above 8 mg/L (according to viral epidemiological profile 112). Nutrient amounts 144 may then be calculated as, for instance using banding, where each datum of viral epidemiological profile 112 elicits a particular numerical value range of nutrient amount 144 or combinations of amounts. In non-limiting illustrative examples, such a calculation may include querying for and retrieving a standard amount of water soluble vitamins for a healthy adult, for instance as described below in Table 1:

TABLE 1

| Nutrient | Amount |
|---|---|
| Vitamin C | 60 mg/day |
| Thiamin (B1) | 0.5 mg/1,000 kcal; 1.0 mg/day |
| Riboflavin (B2) | 0.6 mg/1,000 kcal; 1.2 mg/day |
| Niacin (B3) | 6.6 NE/1,000 kcal; 13 ND/day |
| Vitamin B6 | 0.02 mg/1 g protein; 2.2 mg/day |
| Vitamin B12 | 3 μg/day |
| Folic Acid | 400 μg/day |

Continuing in reference to FIG. 1, in reference to Table 1 above, wherein NE is niacin equivalent (1 mg niacin, or 60 mg tryptophan), mg (milligram), kcal (1000 kcal=1 Calorie), and μg (microgram). Computing device 104 may store and/or retrieve the above standard nutrient amounts, for instance in a database. The amounts may be re-calculated and converted according to a user's viral epidemiological profile 112. For instance, these amounts may relate to an average BMI, adult male, in a heavily populated area experiencing high numbers of Rotavirus. In this case, the amounts may be adjusted according to unique user-specific viral epidemiology profile 112, and an equation derived by machine-learning may be used to “learn” which amounts to increase, and which to decrease. In non-limiting illustrative examples, an obese woman who was recently placed on a 1,400 Calorie/day diet, and not may require the above amounts and per-user amounts may be recalculated according to such a diet, where some amounts may increase, some may decrease, and some may remain constant. For instance, if such a person were prone to viral infection due to nutrition deficiency, a particular increase among vitamin C, zinc, vitamin E, and vitamin A may be calculated according to a weighting factor associated with such a pattern of input data.

Continuing in reference to FIG. 1, calculating nutrient amounts 144 may include deriving a weighting factor to adjust, or otherwise re-calculate, an amount. Weighting factor may be determined by computing device 104, for instance, by querying for vitamin amounts according to data inputs identified in the viral epidemiological profile 112.

Continuing in reference to FIG. 1, calculating nutrient amounts 144 may include generating training data using the plurality of effects of the plurality of nutrient amounts 144 identified according to the viral infection category 124. For instance and without limitation, generating training data may include searching, using a web browser and the Internet, to locate nutrient amount 144 relating to viral infection category 124. Generating training data may include retrieving effect output by spread model 140. Training data for deriving nutrient amounts 144 may include viral spread training data 136. Viral spread training data 136 for calculating nutrient amounts 144 may include immunological, clinical, and viral infection pathology outcomes as a function of nutrient supplementation in a variety of specifics cases (cohort age, virus type, time of year, starting deficiency level, etc.).

Continuing in reference to FIG. 1, calculating nutrient amounts 144 may include training a nutrition machine-learning model according to the training data, wherein training data includes a plurality of data entries that correlates the magnitude of nutrient effect to a plurality of nutrient amounts for each viral infection category 124. Nutrition machine-learning model 148 may include any machine-learning process, model, method, and/or algorithm as described herein, that may be performed by machine-learning model, as described in further detail below. Nutrition machine-learning model 148 may be trained with training data, as described herein, to derive a dosage-based effect (mathematical function) for a plurality of nutrients (values) according to viral infection category 124. In such a case the machine-learning model may accept an input of a viral infection category 124, such as an anticipated viral infection, and then output nutrient amounts 144, which may include effects, nutrient identities, nutrient combinations, and specific per-user amounts.

Continuing in refence to FIG. 1, for example in non-limiting illustrative examples, training data for nutrition machine-learning model 148 may include vitamin A supplementation from animal models which may result in higher serum anti-rabies immunoglobin (IG) (2.1 times) than those without supplementation, supporting the supplementation of vitamin A for boosting immunological function in individuals with high rabies incidence. Supplementation of vitamin A in combination with vitamin D in children with insufficient or deficient levels of RBP (vitamin A) and 25-hydroxyvitamin D (vitamin D) may result in greater antibody responses (IG concentration). Such data may be used to derive, for instance and without limitation, a function for a particular increases (e.g., mg/kg body weight) of vitamin A/vitamin D combination dosage and frequency. Training data may include serum levels of the vitamins, and their associated vitamers in the blood (carotenoids, calcitriol, etc.), as a function of supplementation, versus immunological response to virus. Such training data may exist for individual classes of IGs (e.g., IgG, IgM, IgA, etc.), particular viral infection categories 124, age, and the like. Accordingly, training data may be organized by a classifier, which classifies data into subsets according to similarity, cohort grouping, etc., as described in further detail below. Training data may be used, for instance, to train nutrition machine-learning model 148 to derive per-user equations, functions, etc., for calculating efficacious nutrient amounts 144, as it may relate to individual virus families, viral infection categories 124, etc.

Continuing in reference to FIG. 1, in further non-limiting illustrative examples, training data for nutrition machine-learning model 148 may include data describing that significantly more Hepatitis C virus patients (HCV+) were HSV-RNA negative (at week 4, 12 and 24), wherein vitamin D supplementation may have been strongly and independently associated with sustained virological response in multivariate analysis. Additionally, ninety-five percent in the vitamin D supplemented cohort were HCV-RNA negative at week 4 and 12. At 24 weeks (approximately 6 months) sustained virological response was significantly more in supplemented group. In such an example, logistic regression analysis may identify vitamin D supplement as an independent predictor of viral response. Although clinically may not be true for all viral infection categories 124, as no significant difference in incidence of wintertime upper respiratory tract infections may be observed in those with vitamin D supplemented diets than compared to standard amounts of vitamin D. It is thought that higher than recommended dosages may not hold a benefit for the Hepatitis viral infection categories 124, but normal vitamin D supplementation may remain beneficial in those than vitamin D deficiency. Although, vitamin D supplemented cohorts may have shown a higher TGFβ plasma level (viral biomarker 108) in response to influenza vaccination without improved antibody response; vitamin D may have a function in directing lymphocyte (white blood cell) polarization toward a tolerogenic immune response. In such an instance as vitamin D, a relationship may be uncovered using nutrition machine-learning model 148 trained with the above training data, that supplementation of a particular vitamin only holds benefit if a deficiency is found, and otherwise not helpful to supplement at certain times of the year, for certain virus types, or for those with varying degrees of deficiency. Additionally, it may be found that nutrient amounts 144 are highly user-dependent where incidence of virus (such as the case with COVID-19), access to vaccination, weather, climate, population density may all represent confounding variables in any function derived with machine-learning. Such variables may be encapsulated and mathematically described in spread model 140, as spread model 140 informs effects of nutrients for such variables; whereas, calculated nutrient amounts may be generated by nutrition machine-learning model 148.

Continuing in reference to FIG. 1, in additional non-limiting illustrative examples, training data for nutrition machine-learning model 148 may include positive effects of vitamin E supplementation as observed in the treatment of chronic Hepatitis B in a small pilot randomized control trial (RCT), where a significantly higher normalization of liver enzymes (ALT/AST, viral biomarkers 108) and HBV-DNA negativization (viral biomarker 108), may be observed in the vitamin E supplementation group. Similar results may be observed in an RCT in, for instance, the pediatric population, where vitamin E treatment resulted in a higher anti-HBV seroconversion and virological response. However, vitamin E supplementation may not have an effect on the risk of pneumonia in participants with body weight in a range from 70-89 kg, while vitamin E increased the risk of pneumonia in participants with body weight <60 kg, participants with body weight >100 kg, and with smokers aged 50-69 yrs. The harm of vitamin E supplementation, however, may be observed to be restricted to participants with dietary vitamin C intake above the median. Simply increasing supplementation generically among nutrient amounts 144 may not result in improved viral infection prevention, and in some cases may cause harm to the user. In such an instance, training data is particularly important for deriving per-user nutrient amounts 144 to achieve any intended viral infection prevention efficacy. Although rarely is a single nutrient ever being consumed individually. Persons skilled in the art may appreciate that with greater variety of nutrients, supplementation becomes increasingly complex.

Continuing in reference to FIG. 1, in further non-limiting illustrative examples, training data for nutrition machine-learning model 148 may include correction of specific nutrient combination deficiencies (e.g. zinc, selenium sulfide, beta carotene, ascorbic acid, and vitamin E). Supplementation may be observed after 6 months (post supplementation) and may be maintained throughout the first year, during which there may be no effect on delayed-type hypersensitivity (DTH) skin response; however, the number of patients without respiratory tract infections during the supplementation period may be higher in groups that received the combinatorial nutrient supplementation. Immunologically, antibody titers (IG concentration) after influenza vaccination may be higher in groups that received combination trace element supplementation alone or associated with vitamins, whereas the vitamin group (vitamin supplementation alone) may have significantly lower antibody titers. Moreover, neither daily multivitamin mineral supplementation at physiological dose, nor 200 mg of vitamin E (alone), showed a favorable effect on incidence and severity of acute respiratory tract infections in well-nourished, non-institutionalized elderly individuals. Training data in the form of vitamin supplementation versus immunological responses may be used to derive nutrient relationships between combinations of supplements. Such relationships may show that certain nutrients may not need to be increased, or that different nutrients may need to be increased as a function of viral epidemiological profile 112 (age, institutionalization) and viral infection category 124 (antibody type, vaccination possible, etc.).

Continuing in reference to FIG. 1, in non-limiting illustrative examples, vitamin A is a fat-soluble vitamin, which is crucial for maintaining vision, promoting growth and development, and protecting epithelium and mucosal integrity in the body. It is known to play an important role in enhancing immune function and having a regulatory function in both cellular and humoral immune responses. Vitamin A supplementation to infants may show the potential to improve antibody response after some vaccines, including measles and anti-rabies vaccination. Correspondingly, an enhanced immune response to influenza virus vaccination may also be observed in children (2-8 years) who were vitamin A and D-insufficient at baseline, after supplementation with vitamin A and D. Vitamin D, another fat-soluble vitamin, also plays a vital role in modulating both innate and adaptive immune responses. Epidemiological data has linked vitamin D deficiency to increased susceptibility to acute viral respiratory infections. Possible mechanisms may suggest that vitamin D plays an important modulatory role of the innate immune responses to respiratory viral infections, such as Influenza A and B, parainfluenza 1 and 2, and Respiratory syncytial virus (RSV). A systematic review on the role of vitamin D in the prevention of acute respiratory infections, which included 39 studies (4 cross-sectional studies, 8 case-control studies, 13 cohort studies and 14 clinical trials), noted that observational studies predominantly report statistically significant associations between low vitamin D status and increased risk of both upper and lower respiratory tract infections. However, results from RCTs included in the above systematic review were conflicting, possibly, reflecting heterogeneity in dosing regimens and baseline vitamin D status in study populations. Few RCT have been conducted subsequent to the above systematic review. A study on the effect of high-dose (2000 IU/day) vs. standard-dose (400 IU/day) vitamin D supplementation on viral upper respiratory tract infections did not show any significant difference between the two group. However, only about ⅓ of the study population had vitamin D levels <30 ng/ml. Similarly in another RCT, a monthly high-dose (100,000 IU/month) vitamin D supplementation reduced the incidence of acute respiratory infections in older long-term care residents, in comparison to a standard dose group (12,000 IU/month). It is evident that vitamin D supplementation may play a role in antiviral immunity. Furthermore, vitamin D has demonstrated a beneficial effect in other viral infections, for example adding vitamin D to conventional Peg-α-2b/ribavirin therapy for treatment-naïve patients with chronic HCV genotype 1 infection significantly improved the viral response, and a similar effect may also be observed in patients with HCV genotype 2-3.

Continuing in reference to FIG. 1, computing device 104 may calculate nutrient amounts 144 as a function of the nutrition machine learning model 148 and the per-user pharmacokinetics. For instance and without limitation, using training data such as described herein, computing device 104 may calculate nutrient amounts 144 by determining differences in nutrient quality from organic sources (food items) from nonorganic sources (commercially-available supplements) from a bioavailability standpoint. Per-user pharmacokinetics, rates of metabolism and/or adsorption of nutrients amounts 144 may differ among user cohorts, which may negate the effectiveness of proscribing particular diet types and nutrition amounts 144 to users. In such an instance, computing device 104 may account for such details using nutrition machine-learning model 148, trained with training data as described above, to derive equations, functions, and/or mathematical relationships observed in the training data to calculate more accurate and specific nutrient amounts 144.

Continuing in reference to FIG. 1, in non-limiting illustrative examples, nutrition machine-learning model 148 may be used to derive per-user pharmacokinetics of vitamin B6. The machine-learning algorithm may accept training data including a plurality of data values, for instance, the total amount of protein consumed (in grams) and total amount of vitamin B6 consumed (in mg) per day in a diet, and what the serum levels of the vitamin B6 vitamer, pyridoxal-5-phosphate, over the course of a month, and derive the rates of metabolism, or how ‘well’ the user is obtaining the vitamin from nutrition elements and adsorbing vitamin B6. In other words, the machine-learning algorithm may derive a function (e.g. using linear regression, vector quantization, least squares, etc.) that describes the pharmacokinetics for that particular user regarding what amount of vitamin B6 consumed, per amount of dietary protein, results in what corresponding amount of bioactive vitamin compound. Such a mathematical relationship, obtained from a machine-learning model and the training data, may then be used by computing device 104 with an input of the viral epidemiological profile 112, which enumerates the amount viral biomarkers 108 (e.g. cytokine level) and/or incidence of infection, spread rates, etc., to calculate an output which is a more accurate, customized, per-user nutrient amount 144 of vitamin B6 that is curated to specifically prevent viral infection, reduce daily risks of infection, etc., according to user-specific data. Persons skilled in the art may appreciate that this process may be repeated and completed for the full spectrum of nutrients, both required as part of a diet and not required as part of a diet.

Continuing in reference to FIG. 1, computing device 104 may calculate nutrient amounts 144 as a function of the nutrition machine learning model 148 and the viral infection category 124. Computing device 104 may ‘learn’ how to more accurately calculate the amounts by which to decrease, supplement, and/or omit items from a viral infection program. Spread model 140 may relate predicted nutrition effects to viral infection category 124 as a function of the epidemiology of a particular virus, or class of viruses. These effects may be generated as labels, identifiers, and/or any other relating data structure that is associated to viral infection category 124. Nutrition machine-learning model 148 may train with training data, such as described herein, which relates viral infection categories 124 (and associated effects) to specific nutrition amounts 144. This way highly correlated effects and accurate nutrient amounts 144 may be derived for each user, regardless of age, lifestyle, virus type, and other epidemiological factors. Such nutrient amounts 144, as derived by system 100, may be stored and/or retrieved from a database. Persons skilled in the art may appreciate that system 100 may generate a highly-specific, yet varied spectrum of personalized nutrient amounts with increased participation among users.

Continuing in reference to FIG. 1, calculating personalized nutrient amounts 144 may include using nutrition machine-learning model 136 to generate a function (or series of functions) describing nutrient amounts 144 calculated prior to classification to viral infection category 124. In non-limiting illustrative examples, it may be shown that fiber content, which is oftentimes classically reported in a generic sense as “carbohydrates”, is important for particular gastrointestinal viral infections. Patterns may identify, for instance, that plant-based diets, supplemented with particular bacterial species of probiotics may result in personalized nutrient amounts 144 for viral epidemiological profiles 112 classified to those viral infection categories 124. Such a relationship found in training data may be derived and captured in a mathematical relationship, such as a function or equation, and stored prior to any proper “gastrointestinal viral infection” classification has been made. This way, nutrient amounts 144, and corresponding nutrition elements 120 may be retrieved, for instance using a classification algorithm, by retrieving data once a user has been classified to such a viral infection category 124.

Continuing in reference to FIG. 1, calculating nutrient amounts 144 may include calculating nutrient amounts 144 as a function of the nutrition machine learning model 148 and the viral infection category 124. Trained nutrition machine learning model 148 may accept an input of viral epidemiological profile 112 (and associated viral infection category 124) to output nutrient amounts 144. Nutrient amounts 144 may be calculated using a variety of functions, systems of equations, and the like, derived from mathematical relationships and/or heuristics identified in training data, for instance from nutrition elements 120 identified directly from viral infection categories 124. Persons skilled in the art may appreciate that each viral infection category 124, of the 100's+ different types of viral infections, may have a unique algorithm for calculating nutrient amounts 144, of the 100's+ of distinct nutrients (and combinations thereof) identified. For instance and without limitation, each virus type, tissue/organ type, stage of viral outbreak, age of person, viral biomarker 108, viral epidemiological profile 112, etc., may elicit a different mathematical equation for calculating vitamin C. Wherein, vitamin C is one of many water-soluble vitamins, and that each vitamin of that class may have a different equation associated with calculating nutrient amounts 144. Each equation may be derived by nutrition machine learning model 148 according to the training data. Additionally, each user's specific pharmacokinetics, current dietary patterns, and the like, may add a unique step in the calculation, wherein the calculated nutrient amount 144 is further personalized.

Continuing in reference to FIG. 1, identifying the plurality of nutrition elements 120 includes identifying, as a function of the plurality of nutrient amounts 144, the plurality of nutrition elements 120, wherein the plurality of nutrition elements 120 are intended to prevent viral infection as a function of the viral infection category 124. Viral epidemiological profile 112 may be associated with user that does not currently have a viral infection belonging to viral infection category 124. In such an instance, nutrition elements 120 may be “personalized” to an individual in that they are intended to prevent, as described above, viral infection in that individual. Nutrition element 120 may prevent viral infection in that they provide a nutrient intended to meet individualized, calculated nutrient amounts 144. Nutrient element 120 may prevent viral infection in addressing nutrient deficiencies, surplus, and the like. Nutrition element 120 may include foods and supplements intended to address genetic and viral biomarker 108 issues that are unique to each individual. “Curating” nutritional elements 120, as used in this disclosure, is a process of combining ingredients and/or nutrients according to calculated nutrient amounts 144. Curated nutritional elements 120 may include combining ingredients such as spices, plant-based materials, animal products, probiotic cultures, and the like, to result in a custom nutritional element 120, such as a particular “health shake”, unique dish, or the like, that may not be available commercially.

Continuing in reference to FIG. 1, computing device 104 is configured to identify, as a function of the calculated nutrient amounts 144, the plurality of nutrition elements 120, wherein the plurality of nutrition elements 120 may be intended to address a datum in the viral epidemiological profile 112. Nutrition elements 120, “intended to address a datum in the viral epidemiological profile 112,” may refer to the process(es) of viral infection treatment, recovery, and/or prevention. “Viral infection treatment,” as used in this disclosure, is the amelioration of viral infection symptomology; such as nutrition elements 120 intended for a person with fever, cough, runny nose, chills, etc. “Viral infection prevention,” as used in this disclosure, is the reduction in risk for viral infection. Viral infection prevention may include specifically curated nutrition elements 120 according to relationships regarding the risk of viral infection, wherein the risk may be decreased if nutrient targets are achieved, and risk may increase quicker then expected according to epidemiological factors.

Continuing in reference to FIG. 1, identifying the plurality of nutrition elements 120 includes identifying the nutrition elements 120 according to the viral infection category 124. Identifying nutrition element 120 according to viral infection category 124 may include querying, for instance using a web browser and the Internet, for foods, supplements, bioactive ingredients, and the like, which are correlated with a particular viral infection category 124. For instance and without limitation, computing device 104 may organize a search for nutrition elements 120 intended for “Cardiovascular Viral Infection”, wherein an entire diet may be crafted around target nutrient amounts 144 and the categorization of the viral epidemiological profile 112 to “Cardiovascular Viral Infection”. In such an example, the nutrition elements 120 are outputs generated from an input search criteria of “Cardiovascular Viral Infection”. The output elements become “personalized” as they are arranged into daily, weekly, monthly, etc., individual meals and/or meal schedule according to a user's particular calculated nutrient amounts 144. The viral infection category 124 may serve as a filtering step, wherein a search is guided by the viral epidemiological profile 112 as it was classified to a viral infection category 124.

Continuing in reference to FIG. 1, computing device 104 may identify the plurality of nutrition elements 120 by using nutrient amount 144 as an input and generating combinations, lists, or other aggregates of nutrition elements 120 necessary to achieve nutrient amount 144. For instance, computing device 104 may use a template nutrient amount 144 of ‘200 mg vitamin C’ and build a catalogue of nutritional elements 120 until the 200 mg vitamin C value is obtained. Computing device 104 may perform this task by querying for food items, for instance from a menu, grocery list, or the like, retrieving the vitamin C content, and subtracting the value from the nutrient amount 144. In non-limiting illustrative examples, computing device 104 may identify orange juice (90 mg vitamin C/serving; 200 mg−90 mg=110 mg) for breakfast, Brussel sprouts (50 mg vitamin C/serving; 110 mg−50 mg=60 mg) for lunch, and baked potato (20 mg vitamin C/serving) and spicy lentil curry (40 mg vitamin C/serving; 60 mg−(20 mg+40 mg)=0 mg) for dinner. In such an example, computing device 104 may search according to a set of instructions (e.g. food preferences, allergies, restrictions, etc.) present in a viral epidemiological profile 112, provided by a physician, user, or the like, and subtract each identified nutrition element 120 nutrient amount from nutrient amount 144 until a combination of nutritional elements 120 that represents a solution is found. Once a solution is found, computing device 104 may generate a file of nutrition elements 120 and store in a database, as described in further detail below.

Continuing in reference to FIG. 1, generating combinations of nutrition elements 120 to achieve nutrient amounts 144 may include generating an objective function. An “objective function,” as used in this disclosure, is a mathematical function that may be used by computing device 104 to score each possible combination of nutrition elements 120, wherein the objective function may refer to any mathematical optimization (mathematical programming) to select the ‘best’ element from a set of available alternatives. Selecting the ‘best’ element from a set of available alternatives may include a combination of nutrition elements 120 which achieves the nutrient amounts 144 in addressing viral epidemiological profile 112 in a user.

Continuing in reference to FIG. 1, in non-limiting illustrative examples, an objective function may include performing a greedy algorithm process. A “greedy algorithm” is defined as an algorithm that selects locally optimal choices, which may or may not generate a globally optimal solution. For instance, computing device 104 may select combinations of nutrition elements 120 so that values associated therewith are the best value for each category. For instance, in non-limiting illustrative example, optimization may determine the combination of the most efficacious ‘serving size’, ‘timing of consumption’, ‘probiotic product’, ‘vegetable’, etc., categories to provide a combination that may include several locally optimal solutions but, together, may or may not be globally optimal in combination.

Still referring to FIG. 1, in further non-limiting illustrative examples, objective function may be formulated as a linear objective function, which computing device 104 may solve using a linear program, such as without limitation, a mixed-integer program. A “linear program,” as used in this disclosure, is a program that optimizes a linear objective function, given at least a constraint; a linear program may be referred to without limitation as a “linear optimization” process and/or algorithm. For instance, in non-limiting illustrative examples, a given constraint might be a metabolic disorder of a user (e.g. lactose intolerance, poor absorption, food allergy, user preference, etc.), and a linear program may use a linear objective function to calculate ingredient combinations, considering how these limitations effect combinations. In various embodiments, system 100 may determine a set of instructions towards addressing a subject's viral epidemiological profile 112 that maximizes a total viral infection prevention score subject to a constraint that there are other competing objectives. For instance, if achieving one nutrient amount 144 by selecting from each nutrition element 120 may result in needing to select a second nutrition element 120, wherein each may compete in viral infection prevention (e.g. adopting two or more diet types simultaneously may not be feasible, a vegan option and a non-vegan option, etc.). A mathematical solver may be implemented to solve for the set of instructions that maximizes scores; mathematical solver may be implemented on computing device 104 and/or another device in system 100, and/or may be implemented on third-party solver.

With continued reference to FIG. 1, in further non-limiting illustrative examples, objective function may include minimizing a loss function, where a “loss function” is an expression of an output which a process minimizes to generate an optimal result. For instance, achieving nutrient amounts 144 may be set to a nominal value, such as ‘100’, wherein the objective function selects elements in combination that reduce the value to ‘0’, wherein the nutrient amounts 144 are ‘100% achieved’. In such an example, ‘maximizing’ would be selecting the combination of nutrition elements 120 that results in achieving nutrient amounts 144 by minimizing the difference, where suboptimal pairing results in score increases. As a non-limiting example, computing device 104 may assign variables relating to a set of parameters, which may correspond to viral infection prevention components, calculate an output from a mathematical expression using the variables, and select an objective that produces an output having the lowest size, according to a given definition of “size.” Selection of different loss functions may result in identification of different potential combinations as generating minimal outputs, and thus ‘maximizing’ efficacy of the combination.

Continuing in reference to FIG. 1, computing device 104 is configured to generate, using the plurality of nutrition elements 120, the viral alleviation program. A “alleviation program,” as used in this disclosure, is a collection of nutrient amounts 144 and nutrition elements 120 organized into a frequency (timing) and dosage (serving size) schedule. Alleviation program 152 may include a remedy after viral infection. Alleviation program 152 may include a nutrition element intended to prevent viral infection as a prophylaxis. Alleviation program 152 may include gathering, classifying, or otherwise categorizing nutrient amounts 144, nutrition elements 120 lists, or the like, which incorporates virus-specific recommendations. For instance, nutrition elements 120 may be scored with a numerical score scale that associates a meal, beverage, supplement, etc., with preventing infection, benefit to virus-infected patient, and the like. Alleviation program 152 may include selecting nutrition elements 120 according to a threshold score, where items above the threshold are selected and arranged. Threshold score may include a daily threshold, wherein nutrition elements 120 are selected each day according to the threshold; and threshold may include a numerical value relating to infection prevention, nutrient amount 144, among other outputs of system 100 described herein. Determining alleviation program 152 may include machine-learning. For instance, training a machine-learning model to identify a scoring rubric for building the alleviation program 152 based on some criteria such as infection prevention, minimizing daily risk, in response to viral outbreak, epidemiological factors, spread model 140, among other criteria. Alleviation program 152 may relate specific viral strains to specific nutrients of interest and provide nutrition element 120 scheduling times and serving sizes for each meal. Alleviation program 152 may differ from one user to the next according to the magnitude of the disease outline (viral infection category 124 and viral epidemiological profile 112).

Continuing in reference to FIG. 1, generating the viral alleviation program 152 may include generating an alleviation program classifier using a nourishment classification machine-learning process to classify the plurality of nutrient amounts 144 to the plurality of nutrition elements 120, and outputting the plurality of nutrition elements as a function of the alleviation program classifier. Alleviation program classifier 156 may include any machine-learning model generated by a classification machine-learning process, as described herein, performed by a machine-learning module as described in further detail below. Training data for alleviation program classifier 156 may include any training data, as described herein, which may be used to classify nutrient amounts 144 to nutrition elements 120. Such training data may include nutrition facts of food items, supplements, etc., which may be modified by per-user pharmacokinetics to derive product-specific nutrient amounts 144 for each nutrition element 120. Alleviation program classifier 156 may accept an input of nutrient amounts 144 and output a plurality of nutrition elements 120 with associated frequency (timing) and dosage (serving size) schedule according to relationships between nutrition elements 120 and nutrient amounts 144. For instance and without limitation, alleviation program classifier 156 may contain relationships between individual fruits and vegetables, that when more vegetables are selected, certain fruits may not be necessary to schedule within the same timeframe (day, meal, etc.). Such a classification process may determine a function, system of equations, and the like, which may be solved for in determining which nutrition elements 120 (fruits, vegetables, meats, dairy, grains, etc.) are useful to obtaining the nutrient amounts 144, while not missing some lower limits of nutrient amounts 144 (trace elements) and not exceeding upper limits for other nutrient amounts 144 (calories).

Continuing in reference to FIG. 1, alleviation program 152 may include a recommended nutrition plan and a recommended supplement plan that at least addresses viral biomarker 108, mitigates symptoms, side-effects, etc. Alleviation program 152 may contain a plan with timing of meals, calorie amounts, vitamin amounts, mineral amounts, etc. Alleviation program 152 may include food items combined with a supplement of non-food items. Alleviation program 152 may be presented as a function of reversing, treating, and/or preventing viral infection for non-infected individuals, for instance an otherwise healthy person to reduce their current risk during an outbreak.

Continuing in reference to FIG. 1, generating the viral alleviation program 152 may include generating a viral prevention metric, wherein the viral prevention metric reflects the level of user participation in the viral alleviation program 152. A “viral prevention metric,” as used in this disclosure, is a numerical value, metric, parameter, and the like, described by a function, vector, matrix, or any other mathematical arrangement, which enumerates a user's current viral infection risk as a function of their level of participation in viral alleviation program 152. Viral prevention metric 160 may include using a machine-learning process, algorithm, and/or model to derive a numerical scale along which to provide a numerical value according to a user's viral epidemiological profile 112 and participation in alleviation program 152 generated from viral epidemiological profile 112. For instance, such a machine-learning model may be trained with training data, wherein training data contains data entries of nutrient amounts 144 correlated to viral infection prevention. Such a machine-learning model with said training data may be used by computing device 104 to relate the consumption of particular foods in alleviation program 152, to achieving some level of nutrient amount 144, and how the nutrient amount 144 relates to viral infection treatment and prevention, achieving remission, maintaining remission, etc. The current risk of infection may be enumerated in viral prevention metric 160. Such a viral prevention metric 160 may include a score that increases with participation in alleviation program 152 and/or decreases by falling short of nutrient amounts 144. Alleviation program 152 may include one or more treatment plans that incorporate, for instance and without limitation, large quantities of acai berry and other antioxidants, phytonutrients, and bioactive ingredients to prevent oxidative damage that leads to the presence of free radicals. Alleviation program 152 may be focused on mitigating tissue damage due to viral infection for an infected patient, where viral prevention metric 160 is tied to treatment, increasing with achieving full recovery, and again increasing with each timepoint a user remains uninfected.

Continuing in reference to FIG. 1, in non-limiting illustrating examples, falling short of vitamin E and vitamin K nutrient amounts 144, may have a particular effect on viral prevention metric 160 for an individual who has been classified to “skin viral infection” viral infection category 124. Where, chronically falling short of the nutrient amount 144 results in a (−3 score) each month but falling within the nutrient amount 144 range for those two nutrients affords (+1 score for each) every month; the target amount for the preceding month may dictate the score change for each subsequent month. In such a case, a machine-learning model may derive an algorithm which dictates the amount to increase/decrease viral prevention metric 160 for that particular viral infection category 124 according to the nutrient amounts 144. In this case, the machine-learning model is trained to identify the relationship between nutrient amounts 144 and effect on viral infection prevention to derive an equation that relates scoring criteria. The score is then calculated using the model and nutrition data as an input. “Nutrition data,” as used in this disclosure, is data describing consumption by the user. Consumption by the user may include amounts and identities of nutrition elements 120. In this way, system 100 may calculate a viral prevention metric 160 as a function of a user's participation in alleviation program 152, where viral prevention metric 160 is updated with each nutrition element 120 consumed by user.

Continuing in reference to FIG. 1, generating the viral alleviation program 152 may include calculating a change in incidence of viral infection as a function of adhering to alleviation program 152. Calculating a change in incidence of viral infection may include receiving nutritional input from a user, for instance and without limitation, as described in Ser. No. 16/911,994, filed Jun. 25, 2020, titled “METHODS AND SYSTEMS FOR ADDITIVE MANUFACTURING OF NUTRITIONAL SUPPLEMENT SERVINGS,” the entirety of which is incorporated herein by reference. Nutritional input of a user may include a designation of any nutrition elements 120 user may have consumed. Nutritional elements 120 may have nutrient amounts 144 associated therewith, which may be applied to a user's current viral epidemiological profile 112, viral infection category 124, and the like. Applying the nutrient amounts 144 may include calculating a difference in viral prevention metric 160. Applying the nutrient amounts 144 may include calculating a change in viral infection risk, likelihood, or incidence as a function of achieving nutrient amounts 144, as described above.

Continuing in reference to FIG. 1, generating the viral alleviation program 152 may include receiving a user preference regarding the plurality of nutrition elements 120, and modifying the plurality of nutrition elements 120 as a function of the user preference. A “user preference,” as used in this disclosure, is a user input that designates a preference related to at least a nutrition element 120. User preference may include designations of nutrition elements 120 to avoid and/or include such as particular food groups, condiments, spices, dietary restrictions such as no animal products, cuisine type such as Mediterranean foods, time of day for eating such as fasting before 10 am, etc. In this way, computing device 104 may accept an input of user preference filter, sort, classify, or otherwise modify the data structure of nutrition elements 120 and schedule the nutrition elements 120 into alleviation program 152 in a custom, per-user manner. Computing device 104 may modify the plurality of nutrition elements 120 as a function of the user preference, for instance by providing recipes with steps omitted, new steps added, or entirely new recipes altogether utilizing the same or different nutrition elements 120. Computing device 104 may modify the plurality of nutrition elements 120 as a function of the user preference by generating a new file, based on the preference, and storing and/or retrieving the file from a database, as described in further detail below.

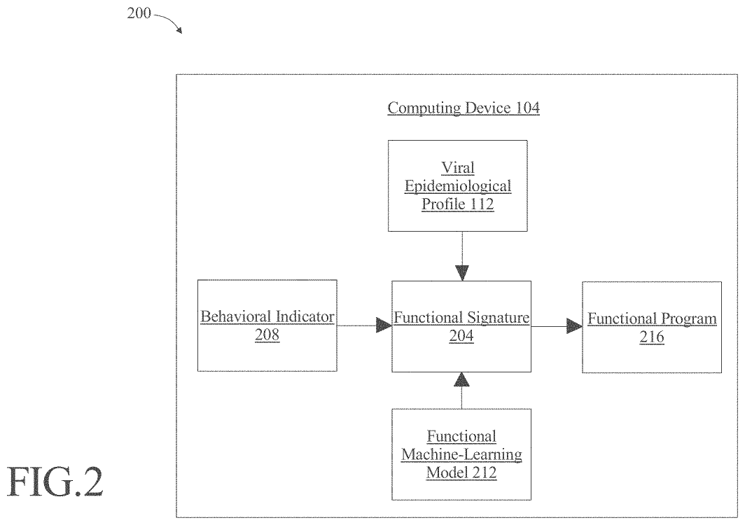

Now referring to FIG. 2, an exemplary embodiment of a system 200 for generating an integrative program is illustrated. System 200 includes computing device 104. Computing device 104 may include any computing device 104 as described above in detail, in reference to FIG. 1. For example and without limitation, computing device 104 may include a microcontroller, microprocessor, digital signal processor (DSP) and/or system on a chip (SoC) as described in this disclosure. an exemplary embodiment of a method 800 for generating a viral epidemiologic disease integrative program is illustrated. Computing device 104 is configured to retrieve a viral epidemiological profile 112 related to the user. Viral epidemiological profile 112 includes any of the viral epidemiological profile 112 as described above, in reference to FIG. 1. In an embodiment, and without limitation, producing viral epidemiological profile 112 may further comprise identifying a contagion element. As used in this disclosure a “contagion factor” is a quantitative value denoting the contagiousness of a viral agent. In an embodiment and without limitation, contagion factor may include a basic reproduction number, R₀. As used in this disclosure a “basic reproduction number” is the expected number of cases directly generated by one case in a population where all individuals are susceptible to infection. For example, and without limitation, Ebola may have a R₀ of 1.5-1.9. As a further non-limiting example, HIV/AIDS may have an R₀ of 2-5. For example, and without limitation a contagion factor may be less than 1, wherein a value less than 1 denotes that each infected individual may infect less than one new individual. As a further non-limiting example, a contagion factor may equal to 1, wherein a value of 1 denotes that each infected individual may infect one new individual. As a further non-limiting example, a contagion factor may greater than 1, wherein a value greater than 1 denotes that each infected individual may infect more than one new individual.

Still referring to FIG. 2, computing device 104 is configured to identify an integrative signature 204 as a function of viral epidemiological profile 112. As used in this disclosure a “integrative signature” is a profile representing an individual's relative measure of wellness. For example and without limitation, integrative signature 204 may represent that an individual is “healthy” and/or in an excellent wellness state. As a further non-limiting example, integrative signature 204 may represent that an individual has is “unhealthy” and/or in a poor wellness state. Integrative signature 204 is identified as a function of receiving a behavioral indicator 208. As used in this disclosure a “behavioral indicator” is an element of data denoting an individual's lifestyle choices. In an embodiment behavioral indicator 208 may include one or more biological, psychological, social, and/or spiritual elements. For example, and without limitation, behavioral indicator 208 may denote a biological element, wherein the biological indicator may denote that an individual has low cholesterol and/or exercises frequently. As a further non-limiting example, behavioral indicator 208 may denote a psychological element, wherein the psychological element may denote that an individual is happy and/or content. As a further non-limiting example, behavioral indicator 208 may denote a social element, wherein the social element may indicate that an individual has 36 friends. As a further non-limiting example, behavioral indicator 208 may denote a spiritual element, wherein the spiritual element may indicate that an individual belongs to the Hinduism religion. As a further non-limiting example, spiritual element may denote one or more chakras and/or spiritual energies of an individual. In an embodiment behavioral indicator 208 may denote one or more lifestyles groups such as, but not limited to, general lifestyles, income, profession, and/or occupation lifestyles, consumption-based lifestyles, social and/or political lifestyles, marketing lifestyles, military lifestyles, sexual lifestyles, spiritual lifestyles, religious lifestyles, musical lifestyles, recreational lifestyles, and the like thereof. For example, and without limitation, lifestyles may include activism, asceticism, modern primitivism, bohemianism, communal living, clothes free, groupie lifestyle, hippie, quirkyalone, rural lifestyle, simple living, traditional lifestyle, criminality, farming, jet set, piracy, poverty, prostitution, sarariman, workaholic, yuppie, social liberalism, social conservatism, polygamy, monogamy, ahimsa, Hinduism, Christianity, evangelicalism, Islam, Judaism, missionary, Zen, yoga, Thelema, surfer, athleticism, hunter, artist, golf, recreational drug use, and the like thereof. Additionally or alternatively behavioral indicator 208 may include one or more markers associated with an individual's behavior such as, but not limited to, markers associated to genetics test data. For example, and without limitation markers may include but are not limited to biological samples, biomarkers, genetic test data, and the like thereof as defined above, in reference to FIG. 1.

Still referring to FIG. 2, behavioral indicator 208 may include a dimensional element. As used in this disclosure a “dimensional element” is an element of datum denoting a relative measure of wellness of an individual. For example, and without limitation dimensional element may denote one or more dimensions associated with healthy living. In an embodiment dimensional element may include an occupational dimension. As used in this disclosure an “occupational dimension” is a dimension of wellness representing personal satisfaction and enrichment in an individual's life through work and/or occupation. For example, and without limitation, occupational dimension may denote that an individual's job is rewarding due to the contribution of personal values, interests, and/or beliefs that are shared among the job and the individual. In an embodiment dimensional element may include a physical dimension. As used in this disclosure a “physical dimension” is a dimension of wellness representing physical activity and/or nutrition. For example, and without limitation, physical dimension may include a dimension associated with eating whole grain foods and/or lean protein foods diet and/or nutrition, while concurrently discouraging the use of recreational drugs. As a further non-limiting example, physical dimension may include a dimension associated with regular exercise and/or enhanced physical strength. In an embodiment dimensional element may include a social dimension. As used in this disclosure a “social dimension” is a dimension of wellness representing an individual's contributions towards the environment and/or community. For example, and without limitation, social dimension may include a dimension associated with an individual's contributions towards the common welfare of the community and/or living in harmony with other.

In an embodiment and still referring to FIG. 2, dimensional element may include an intellectual dimension. As used in this disclosure a “intellectual dimension” is a dimension of wellness representing an individual's creative and/or mental activities. For example, and without limitation, intellectual dimension may include a dimension associated with an individual's abilities to identify potential problems and choose appropriate courses of action based on available information than to wait, worry, and contend with major concerns later. In an embodiment dimensional element may include a spiritual dimension. As used in this disclosure a “spiritual dimension” is a dimension of wellness representing an individual's search for meaning and/or purpose of existence. For example, and without limitation, spiritual dimension may include a dimension associated with an individual's understanding of the meaning for existence and/or the tolerance of other's meaning for existence. In an embodiment dimensional element may include an emotional dimension. As used in this disclosure an “emotional dimension” is a dimension of wellness representing an individual's awareness and/or acceptance of feelings. For example, and without limitation, emotional dimension may include a dimension associated with an individual's feelings related to a belief, philosophy, behavior, and the like thereof.

Still referring to FIG. 2, computing device 104 may receive behavioral indicator 208 as a function of obtaining an exposure element. As used in this disclosure an “exposure element” is an element of datum representing contact and/or exposure associated with a lifestyle. For example, and without limitation exposure element may denote prolonged contact to radioactive material as a function of being a nuclear power plant technician. As a further non-limiting example exposure element may denote prolonged contact to illicit drugs as a function of being a recreational drug user. As a further non-limiting example, exposure element may denote prolonged contact to heavy metals in water as a function of having a surfing lifestyle. In an embodiment, and without limitation, exposure element may denote one or more exposures to toxins such as, but not limited to, persistent organic pollutants, polychlorinated bisphenols, hydrogen chlorides, benzenes, xylenes, toluenes, dioxins, heavy metals, radioactivity, and the like thereof. In another embodiment, exposure element may denote one or more epigenetic factors. As used in this disclosure an “epigenetic factor” is a factor denoting a likelihood of a change in gene activity and/or expression as a function of one or more external factors. For example, and without limitation, epigenetic factor may denote a high likelihood for a gene mutation as a function of a polyaromatic hydrocarbon. As a further non-limiting example, epigenetic factor may denote a high likelihood for reduced gene expression as a function of aluminum toxicity and/or poisoning.

In an embodiment, and still referring to FIG. 2, integrative signature 204 may be obtained as a function of obtaining a salubrious reference. As used in this disclosure a “salubrious reference” is a guideline and/or recommendation representing an ideal health level of an individual. For example, and without limitation salubrious reference may include a guideline that a viral epidemiologic profile should maintain a body temperature of 98.7° F. As a further non-limiting example, salubrious reference may denote that an individual should exercise for 30 minutes every other day. As a further non-limiting example, salubrious reference may denote that an individual should attend a religious gathering once a week. As a further non-limiting example, salubrious reference may denote that an individual should meditate twice a day for 10 minutes. As a further non-limiting example, salubrious reference may denote that an individual should have 5 or more chakras balanced during a particular time period, wherein a time period includes milliseconds, seconds, minutes, hours, days, weeks, months, years, and the like thereof. Salubrious reference may be obtained as a function of one or more informed advisors, wherein an informed advisor is described above in detail. Additionally or alternatively, salubrious reference may be obtained as a function of one or more integrative advisors. As used in this disclosure a “integrative advisor” is an individual capable of recommending and/or guiding an individual towards a more suited wellness state. For example, and without limitation, integrative advisor may include one or more nutritionists, personal trainers, physical therapists, spiritual leaders, religious leaders, massage therapists, spiritual therapists, reiki masters, acupuncturists, life coaches, priests, philosophers, theologists, yoga instructors, wellness instructors, teachers, and the like thereof. In an embodiment, salubrious reference may include recommendations from one or more medical sources such as peer reviews, informed advisor associations, medical websites, medical textbooks, religious books, prophecies, spiritual texts, and the like thereof.

Still referring to FIG. 2, computing device 104 identifies integrative signature 204 as function of behavioral indicator 208 and viral epidemiological profile using an integrative machine-learning model 212. As used in this disclosure a “integrative machine-learning model” is a machine-learning model that identifies an integrative signature output given viral epidemiological profiles and behavioral indicators as inputs; this is in contrast to a non-machine learning software program where the commands to be executed are determined in advance by a user and written in a programming language. Integrative machine-learning model may include one or more integrative machine-learning processes such as supervised, unsupervised, or reinforcement machine-learning processes that computing device 104 and/or a remote device may or may not use in the determination of integrative signature 204, wherein a remote device is an external device to computing device 104 as described above in detail. A integrative machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 2, computing device 104 may train integrative machine-learning process as a function of an integrative training set. As used in this disclosure a “integrative training set” is a training set that correlates a viral epidemiological profile and a behavioral indicator to an integrative signature. For example, and without limitation, a viral epidemiological profile of measles and a behavioral indicator associated with a user that smokes marijuana daily may relate to an integrative signature of a relatively “unhealthy” wellness state. The integrative training set may be received as a function of user-entered valuations of viral epidemiological profiles, behavioral indicators, and/or integrative signatures. Computing device 104 may receive integrative training set by receiving correlations of viral epidemiological profiles and/or behavioral indicators that were previously received and/or determined during a previous iteration of determining integrative signatures. The integrative training set may be received by one or more remote devices that at least correlate a viral epidemiological profile and behavioral indicator to an integrative signature, wherein a remote device is an external device to computing device 104, as described above. Integrative training set may be received in the form of one or more user-entered correlations of a viral epidemiological profile and/or behavioral indicator to an integrative signature. Additionally or alternatively, a user may include, without limitation, an informed advisor and/or an integrative advisor entering correlations of viral epidemiological profiles and/or behavioral indicators to integrative signatures, wherein informed advisors and/or integrative advisors may include, without limitation, physicians, nutritionists, therapists, spiritual leaders, and the like thereof as described above in detail.

Still referring to FIG. 2, computing device 104 may receive integrative machine-learning model 212 from a remote device that utilizes one or more integrative machine learning processes, wherein remote device is described above in detail. For example, and without limitation, remote device may include a computing device, external device, processor, and the like thereof. Remote device may perform the integrative machine-learning process using the integrative training set to generate integrative signature 204 and transmit the output to computing device 104. Remote device may transmit a signal, bit, datum, or parameter to computing device 104 that at least relates to integrative signature 204. Additionally or alternatively, the remote device may provide an updated machine-learning model. For example, and without limitation, an updated machine-learning model may be comprised of a firmware update, a software update, an integrative machine-learning process correction, and the like thereof. As a non-limiting example a software update may incorporate a new viral epidemiological profile that relates to a modified behavioral indicator. Additionally or alternatively, the updated machine learning model may be transmitted to the remote device, wherein the remote device may replace the integrative machine-learning model with the updated machine-learning model and determine the physiological as a function of the behavioral indicator using the updated machine-learning model. The updated machine-learning model may be transmitted by the remote device and received by computing device 104 as a software update, firmware update, or corrected integrative machine-learning model. For example, and without limitation an integrative machine-learning model 212 may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, integrative machine-learning model 212 may identify integrative signature 204 as a function of one or more classifiers, wherein a classifier is described above in detail.

Still referring to FIG. 2, computing device 104 may identify integrative signature 204 by producing an indicator index as a function of behavioral indicator 208. As used in this disclosure an “indicator index” is a measurable value associated with a behavioral indicator. For example and without limitation, an indicator index may be 20 for a behavioral indicator associated with meditating 3 times a day for 5 minutes. As a further non-limiting example, an indicator index may be 73 for a behavioral indicator associated with a sedentary lifestyle comprising sitting down for 12 hours a day. In an embodiment, computing device may produce a weighted index as a function of the indicator index and viral epidemiological profile. As used in this disclosure a “weighted index” is a weighted value associated with behavioral indicator and viral epidemiological profile. For example, and without limitation, a behavioral indicator of a lifestyle of smoking tobacco for 30 years may relate to a value of 18, wherein a viral epidemiological profile for chikungunya viras may weight and/or alter the value to adjust to 73.

Still referring to FIG. 2, computing device 104 may identify integrative signature 204 as a function of determining a root cause. As used in this disclosure a “root cause” is a source of origination of a behavioral indicator. For example, and without limitation, root cause may denote that an individual has a sedentary lifestyle as a function of watching television. As a further non-limiting root cause may denote that an individual started smoking as a function of a lack of religious guidance and/or spiritual teaching. As a further non-limiting example, root cause may denote that an individual has emotional instability as a function of one or more traumatic experiences and/or psychological traumas. Additionally or alternatively, computing device 104 may determine a habit as a function of behavioral indicator 208. As used in this disclosure a “habit” is a tendency and/or regularly practiced behavior that an individual performs. For example, and without limitation a habit may include swearing, trichotillomania, picking an individual's nose, smoking cigarettes, biting fingernails, drinking coffee, drinking tea, hair picking, watching television, eating fast food, alcohol, emotional shopping, social media use, drinking soda, eating chocolate, humming, sleeping-in, lying, procrastinating, being unfriendly, and the like thereof.

In an embodiment, and without limitation, identifying integrative signature 204 may further comprise determining a travel routine. As used in this disclosure a “travel routine” is a path and/or route that a user travels and/or moves through over a period of time, wherein period of time is a distance metric used to denote one or more time intervals such as seconds, minutes, hours, days, weeks, months, years, and the like thereof. For example, and without limitation travel routine may denote that a user travels through South Africa every other week. As a further non-limiting example, travel routine may denote that a user moves through one or more cities and/or highways daily. As a further non-limiting example, travel routine may denote that a user travels along one or more public transportation routes, such as but not limited to public transportation routes comprising buses, trains, airplanes, taxi's, ride sharing vehicles, and the like thereof. In an embodiment, and without limitation, determining travel routine may further comprise identifying a geographical prevalence. As used in this disclosure a “geographical prevalence” is a measurable value representing a likelihood for contracting a viral agent as a function of a geographical location, wherein a “geographical location,” as used herein, is a location and/or place used to denote a region on the earth's surface or elsewhere. As a non-limiting example, geographical prevalence may be a value of 80 for a likelihood of contracting Streptococcus pneumoniae as a function of a geographical location of the arctic zone. As a further non-limiting example, geographical prevalence may be a value of 20 for a likelihood of contracting Chagas disease as a function of a geographical location of the temperate zone.

Still referring to FIG. 2, identifying integrative signature 204 further comprises determining a susceptibility element. As used in this disclosure a “susceptibility element” is an element of data representing one or more predispositions and/or susceptibilities that a user may have. For example, and without limitation, susceptibility element may denote that an individual has a predisposition to contracting measles as a function of not having a measles vaccination. As a further non-limiting example, susceptibility element may denote that an individual has a predisposition to contracting pneumonia as a function of living in the arctic zone. In an embodiment, and without limitation, susceptibility element may include an immune element. As used in this disclosure an “immune element” is an element of data representing one or more immune capabilities of a user. For example, and without limitation, immune element may denote that an individual has previously contracted a viral agent, wherein the individual's immune system has immunity for that viral agent. As a further non-limiting example, immune element may denote that an individual has never interacted with a viral agent, wherein the individual's immune system has no immunity for that viral agent. As a further non-limiting example, immune element may denote that an individual has received one or more vaccinations for a viral agent, wherein the user may have immunity for that viral agent. In an embodiment, and without limitation, determining susceptibility element may further comprise identifying a contact element. As used in this disclosure a “contact element” is an element of data representing one or more interactions and/or contacts an individual comprises with another individual over a period of time, wherein a period of time includes a time interval such as seconds, minutes, hours, days, weeks, months, and the like thereof as described above. For example, and without limitation, contact element may denote that an individual has interacted with 4 alternate individuals over the last 2 days. As a further non-limiting example, contact element may denote that an individual has interacted with 30 alternate individuals over the last hour. As a further non-limiting example, contact element may denote that an individual has interacted with 200 alternate individuals over the last 2 years.

Still referring to FIG. 2, computing device 104 is configured to generate an integrative program 216 as a function of integrative signature 204. As used in this disclosure a “integrative program” is a program and/or instruction set to alter an individual's lifestyle to affect viral epidemiological profile and/or integrative signature 204. An integrative program may provide instruction relating to one or more areas of a user's life, including but not limited to, physical fitness, stress management, meditation, spirituality, religion, energy healing, professional endeavors, personal endeavors, body, mind, health, finances, recreation, romance, personal development, and the like. For example, and without limitation, integrative program 216 may include a program that instructs an individual to perform 10 minutes of strenuous exercise every day for 5 weeks. As a further non-limiting example, integrative program 216 may include a program that instructs an individual to meditate for 1 minute every other week. As a further non-limiting example, integrative program 216 may instruct an individual to go on a hike for 2 hours once a week. Additionally or alternatively, integrative program 216 may include an alleviation program 152, wherein an alleviation program 152 is described above in detail, in reference to FIG. 1. For example, and without limitation, integrative program 216 may instruct an individual to consume a paleo diet. In an embodiment and without limitation, integrative program 216 may include one or more instructions such as, but not limited to a first instruction to exercise and a second instruction of alleviation program 152. Computing device 104 may generate integrative program 216 as a function of integrative signature 204 using a program machine-learning model. As used in this disclosure a “program machine-learning model” is a machine-learning model that produces an integrative program output given integrative signatures as inputs; this is in contrast to a non-machine learning software program where the commands to be executed are determined in advance by a user and written in a programming language. Program machine-learning model may include one or more program machine-learning processes such as supervised, unsupervised, or reinforcement machine-learning processes that computing device 104 and/or a remote device may or may not use in the determination of integrative program 216, wherein a remote device is an external device to computing device 104 as described above in detail. A program machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 2, computing device 104 may train program machine-learning process as a function of a program training set. As used in this disclosure a “program training set” is a training set that correlates an integrative signature to an integrative program. For example, and without limitation, an integrative signature of a habit of being exposed to radioactivity may relate to an integrative program of a reduced exposure to radioactivity, exercise for 30 minutes to aid in eliminating the toxin, and increased meditation to reduce inflammation. The program training set may be received as a function of user-entered valuations of integrative signatures, and/or integrative programs. Computing device 104 may receive program training set by receiving correlations of integrative signatures that were previously received and/or determined during a previous iteration of determining integrative programs. The program training set may be received by one or more remote devices that at least correlate an integrative signature to an integrative program, wherein a remote device is an external device to computing device 104, as described above. Program training set may be received in the form of one or more user-entered correlations of an integrative signature to an integrative program. Additionally or alternatively, a user may include, without limitation, an informed advisor and/or an integrative advisor entering correlations of integrative signatures to integrative programs, wherein informed advisors and/or integrative advisors may include, without limitation, physicians, nutritionists, therapists, spiritual leaders, and the like thereof as described above in detail.

Still referring to FIG. 2, computing device 104 may receive program machine-learning model from a remote device that utilizes one or more program machine learning processes, wherein remote device is described above in detail. For example, and without limitation, remote device may include a computing device, external device, processor, and the like thereof. Remote device may perform the program machine-learning process using the program training set to generate integrative program 216 and transmit the output to computing device 104. Remote device may transmit a signal, bit, datum, or parameter to computing device 104 that at least relates to integrative program 216. Additionally or alternatively, the remote device may provide an updated machine-learning model. For example, and without limitation, an updated machine-learning model may be comprised of a firmware update, a software update, a program machine-learning process correction, and the like thereof. As a non-limiting example a software update may incorporate a new integrative signature that relates to a modified integrative program. Additionally or alternatively, the updated machine learning model may be transmitted to the remote device, wherein the remote device may replace the program machine-learning model with the updated machine-learning model and determine the integrative program as a function of the integrative signature using the updated machine-learning model. The updated machine-learning model may be transmitted by the remote device and received by computing device 104 as a software update, firmware update, or corrected program machine-learning model. For example, and without limitation a program machine-learning model may utilize a neural net machine-learning process, wherein the updated machine-learning model may incorporate polynomial regression machine-learning process. Updated machine learning model may additionally or alternatively include any machine-learning model used as an updated machine learning model as described in U.S. Nonprovisional application Ser. No. 17/106,658, the entirety of which is incorporated herein by reference. In an embodiment, and without limitation, program machine-learning model may identify integrative program 216 as a function of one or more classifiers, wherein a classifier is described above in detail.

Still referring to FIG. 2, computing device 104 may generate integrative program 216 as a function of determining a holistic prospect. As used in this disclosure a “holistic prospect” is a potential adjustment to an individual's integrative signature. For example, and without limitation, holistic project may denote that a potential adjustment may include adjusting the amount of exercise and/or strenuous activity performed by the individual. As a further non-limiting example, holistic project may denote that a potential adjustment may include adjusting the amount of religious guidance that an individual receives. As a further non-limiting example, holistic project may denote that a potential adjustment may include adjusting the amount of chakra flow of an individual. As a further non-limiting example, holistic project may denote that a potential adjustment may include adjusting the number of social interactions that an individual experiences each day. As a further non-limiting example, holistic prospect may include a potential adjustment to alleviation program 152 through the alteration of one or more edibles and/or supplementation of alleviation program 152. In an embodiment, holistic prospect may include one or more supplements. For example, and without limitation, a supplement may include vitamin E, linoleic acid, lipoic acid, inositol, magnesium, biotin, progestin, vitamin D, and the like thereof.

Still referring to FIG. 2, computing device 104 generate integrative program 216 as a function of a viral integrative goal. As used in this disclosure a “viral integrative goal” is a predicted goal and/or purposeful plan to modify integrative signature 204 and/or viral epidemiological profile. As a non-limiting example, viral integrative goal may include a treatment goal. As used in this disclosure a “treatment goal” is a viral integrative goal that is designed to at least reverse and/or eliminate integrative signature 204 and/or viral epidemiological profile. As a non-limiting example, a treatment goal may include reversing the effects of adenovirus as a function of exercise, diet, and/or supplementation. As a further non-limiting example, a treatment goal includes reversing SARS as a function of recommending the supplement zinc, recommending edibles such as apples, berries, tomatoes, celery, onions, sauerkraut, kombucha, and the like thereof, recommending a meditation schedule of once per day for 20 minutes, and/or recommending 25 minutes of exercise every other day. Viral integrative goal may include a prevention goal. As used in this disclosure a “prevention goal” is a viral integrative goal that is designed to at least prevent and/or avert integrative signature 204 and/or viral epidemiological profile. As a non-limiting example, a prevention goal may include preventing the development of influenzas as a function of hiking 2 miles per day and/or recommending an alleviation program of a low-carb diet. Viral integrative goal may include a mitigation goal. As used in this disclosure a “mitigation goal” is an integrative goal that is designed to reduce the symptoms and/or effects of a viral epidemiological profile. For example, and without limitation, mitigation goal may include reducing the effects of shingles as a function of recommending magnesium and/or zinc supplements and/or recommending enhanced chakra flow of an individual's body. Additionally or alternatively, viral integrative goal may include one or more goals associated with gene therapy to alter and/or mutate an individual's epigenetic factors.

Still referring to FIG. 2, computing device 104 may generate integrative program 216 as a function of integrative signature 204 and viral integrative goal using a goal machine-learning model. As used in this disclosure a “goal machine-learning model” is a machine-learning model to produce an integrative program output given integrative signatures and/or viral integrative goals as inputs; this is in contrast to a non-machine learning software program where the commands to be executed are determined in advance by a user and written in a programming language. Goal machine-learning model may include one or more goal machine-learning processes such as supervised, unsupervised, or reinforcement machine-learning processes that computing device 104 and/or a remote device may or may not use in the development of integrative program 216. Goal machine-learning process may include, without limitation machine learning processes such as simple linear regression, multiple linear regression, polynomial regression, support vector regression, ridge regression, lasso regression, elasticnet regression, decision tree regression, random forest regression, logistic regression, logistic classification, K-nearest neighbors, support vector machines, kernel support vector machines, naïve bayes, decision tree classification, random forest classification, K-means clustering, hierarchical clustering, dimensionality reduction, principal component analysis, linear discriminant analysis, kernel principal component analysis, Q-learning, State Action Reward State Action (SARSA), Deep-Q network, Markov decision processes, Deep Deterministic Policy Gradient (DDPG), or the like thereof.

Still referring to FIG. 2, computing device 104 may train goal machine-learning process as a function of a goal training set. As used in this disclosure a “goal training set” is a training set that correlates a viral integrative goal to an integrative signature. The goal training set may be received as a function of user-entered integrative signatures, viral integrative goals, and/or integrative programs. For example, and without limitation, a viral integrative goal of treating Chron's disease may correlate to an integrative signature of physical activity and/or a vegan diet. Computing device 104 may receive goal training by receiving correlations of viral integrative goals and/or integrative signatures that were previously received and/or determined during a previous iteration of generating integrative programs. The goal training set may be received by one or more remote devices that at least correlate a viral integrative goal and/or integrative signature to an integrative program, wherein a remote device is an external device to computing device 104, as described above. Goal training set may be received in the form of one or more user-entered correlations of a viral integrative goal and/or integrative signature to an integrative program. Additionally or alternatively, a user may include, without limitation, an informed advisor and/or an integrative advisor entering correlations of integrative signatures to integrative programs, wherein informed advisors and/or integrative advisors may include, without limitation, physicians, nutritionists, therapists, spiritual leaders, and the like thereof as described above in detail.

Still referring to FIG. 2, computing device 104 may receive goal machine-learning model from the remote device that utilizes one or more goal machine learning processes, wherein a remote device is described above in detail. For example, and without limitation, a remote device may include a computing device, external device, processor, and the like thereof. The remote device may perform the goal machine-learning process using the goal training set to develop integrative program 216 and transmit the output to computing device 104. The remote device may transmit a signal, bit, datum, or parameter to computing device 104 that at least relates to integrative program 216. Additionally or alternatively, the remote device may provide an updated machine-learning model. For example, and without limitation, an updated machine-learning model may be comprised of a firmware update, a software update, a goal machine-learning process correction, and the like thereof. As a non-limiting example a software update may incorporate a new viral integrative goal that relates to a modified integrative signature. Additionally or alternatively, the updated machine learning model may be transmitted to the remote device, wherein the remote device may replace the goal machine-learning model with the updated machine-learning model and develop the integrative program as a function of the viral integrative goal using the updated machine-learning model. The updated machine-learning model may be transmitted by the remote device and received by computing device 104 as a software update, firmware update, or corrected goal machine-learning model. For example, and without limitation goal machine-learning model may utilize a neural net machine-learning process, wherein the updated machine-learning model may incorporate decision tree machine-learning processes.

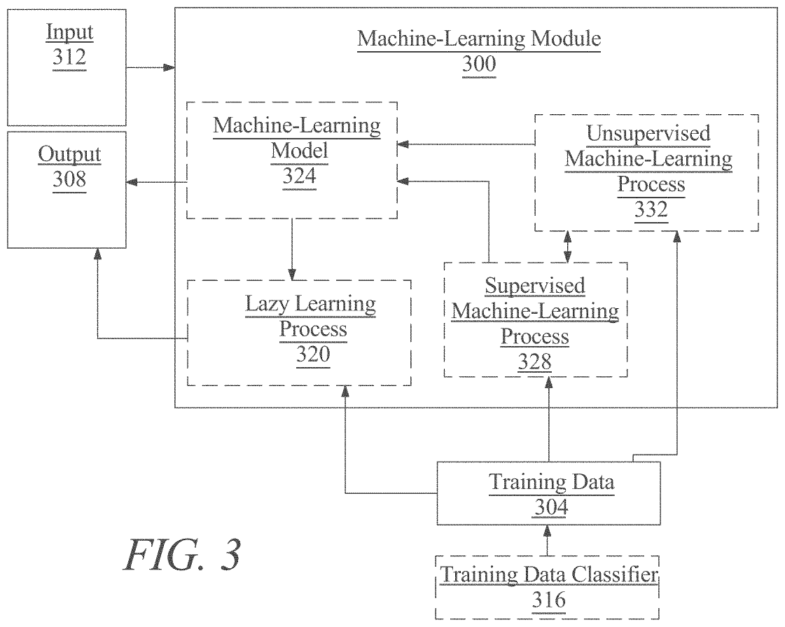

Referring now to FIG. 3, an exemplary embodiment of a machine-learning module 300 that may perform one or more machine-learning processes as described in this disclosure is illustrated. Machine-learning module may perform determinations, classification, and/or analysis steps, methods, processes, or the like as described in this disclosure using machine learning processes. A “machine learning process,” as used in this disclosure, is a process that automatedly uses training data 304 to generate an algorithm that will be performed by a computing device/module to produce outputs 308 given data provided as inputs 312; this is in contrast to a non-machine learning software program where the commands to be executed are determined in advance by a subject and written in a programming language.

Still referring to FIG. 3, “training data,” as used herein, is data containing correlations that a machine-learning process may use to model relationships between two or more categories of data elements. For instance, and without limitation, training data 304 may include a plurality of data entries, each entry representing a set of data elements that were recorded, received, and/or generated together; data elements may be correlated by shared existence in a given data entry, by proximity in a given data entry, or the like. Multiple data entries in training data 304 may evince one or more trends in correlations between categories of data elements; for instance, and without limitation, a higher value of a first data element belonging to a first category of data element may tend to correlate to a higher value of a second data element belonging to a second category of data element, indicating a possible proportional or other mathematical relationship linking values belonging to the two categories. Multiple categories of data elements may be related in training data 304 according to various correlations; correlations may indicate causative and/or predictive links between categories of data elements, which may be modeled as relationships such as mathematical relationships by machine-learning processes as described in further detail below. Training data 204 may be formatted and/or organized by categories of data elements, for instance by associating data elements with one or more descriptors corresponding to categories of data elements. As a non-limiting example, training data 304 may include data entered in standardized forms by persons or processes, such that entry of a given data element in a given field in a form may be mapped to one or more descriptors of categories. Elements in training data 304 may be linked to descriptors of categories by tags, tokens, or other data elements; for instance, and without limitation, training data 304 may be provided in fixed-length formats, formats linking positions of data to categories such as comma-separated value (CSV) formats and/or self-describing formats such as extensible markup language (XML), JavaScript Object Notation (JSON), or the like, enabling processes or devices to detect categories of data.

Alternatively or additionally, and continuing to refer to FIG. 3, training data 304 may include one or more elements that are not categorized; that is, training data 304 may not be formatted or contain descriptors for some elements of data. Machine-learning algorithms and/or other processes may sort training data 304 according to one or more categorizations using, for instance, natural language processing algorithms, tokenization, detection of correlated values in raw data and the like; categories may be generated using correlation and/or other processing algorithms. As a non-limiting example, in a corpus of text, phrases making up a number “n” of compound words, such as nouns modified by other nouns, may be identified according to a statistically significant prevalence of n-grams containing such words in a particular order; such an n-gram may be categorized as an element of language such as a “word” to be tracked similarly to single words, generating a new category as a result of statistical analysis. Similarly, in a data entry including some textual data, a person's name may be identified by reference to a list, dictionary, or other compendium of terms, permitting ad-hoc categorization by machine-learning algorithms, and/or automated association of data in the data entry with descriptors or into a given format. The ability to categorize data entries automatedly may enable the same training data 304 to be made applicable for two or more distinct machine-learning algorithms as described in further detail herein. Training data 304 used by machine-learning module 300 may correlate any input data as described in this disclosure to any output data as described in this disclosure.

Further referring to FIG. 3, training data may be filtered, sorted, and/or selected using one or more supervised and/or unsupervised machine-learning processes and/or models as described in further detail herein; such models may include without limitation a training data classifier 316. Training data classifier 316 may include a “classifier,” which as used in this disclosure is a machine-learning model as defined herein, such as a mathematical model, neural net, or program generated by a machine learning algorithm known as a “classification algorithm,” as described in further detail herein, that sorts inputs into categories or bins of data, outputting the categories or bins of data and/or labels associated therewith. A classifier may be configured to output at least a datum that labels or otherwise identifies a set of data that are clustered together, found to be close under a distance metric as described below, or the like. Machine-learning module 300 may generate a classifier using a classification algorithm, defined as a process whereby a computing device and/or any module and/or component operating thereon derives a classifier from training data 304. Classification may be performed using, without limitation, linear classifiers such as without limitation logistic regression and/or naive Bayes classifiers, nearest neighbor classifiers such as k-nearest neighbors classifiers, support vector machines, least squares support vector machines, fisher's linear discriminant, quadratic classifiers, decision trees, boosted trees, random forest classifiers, learning vector quantization, and/or neural network-based classifiers. As a non-limiting example, training data classifier 316 may classify elements of training data to elements that characterizes a sub-population, such as a subset of viral biomarkers 108 (such as gene expression patterns as it relates to viral epidemiological profile 112) and/or other analyzed items and/or phenomena for which a subset of training data may be selected.

Still referring to FIG. 3, machine-learning module 300 may be configured to perform a lazy-learning process 320 and/or protocol, which may alternatively be referred to as a “lazy loading” or “call-when-needed” process and/or protocol, may be a process whereby machine learning is conducted upon receipt of an input to be converted to an output, by combining the input and training set to derive the algorithm to be used to produce the output on demand. For instance, an initial set of predictions may be performed to cover an initial heuristic and/or “first guess” at an output and/or relationship. As a non-limiting example, an initial heuristic may include a ranking of associations between inputs and elements of training data 304. Heuristic may include selecting some number of highest-ranking associations and/or training data 304 elements, such as classifying nutrition elements 120 to viral infection category 124 elements and assigning a value as a function of some ranking association between elements. Lazy learning may implement any suitable lazy learning algorithm, including without limitation a K-nearest neighbors algorithm, a lazy naïve Bayes algorithm, or the like; persons skilled in the art, upon reviewing the entirety of this disclosure, will be aware of various lazy-learning algorithms that may be applied to generate outputs as described in this disclosure, including without limitation lazy learning applications of machine-learning algorithms as described in further detail herein.

Alternatively or additionally, and with continued reference to FIG. 3, machine-learning processes as described in this disclosure may be used to generate machine-learning models 324. A “machine-learning model,” as used in this disclosure, is a mathematical and/or algorithmic representation of a relationship between inputs and outputs, as generated using any machine-learning process including without limitation any process as described above, and stored in memory; an input is submitted to a machine-learning model 324 once created, which generates an output based on the relationship that was derived. For instance, and without limitation, a linear regression model, generated using a linear regression algorithm, may compute a linear combination of input data using coefficients derived during machine-learning processes to calculate an output datum. As a further non-limiting example, a machine-learning model 324 may be generated by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. A machine-learning model may be used to derive numerical scales for providing numerical values to viral epidemiological profile 112 and/or viral prevention metric 160, etc., as described above, to “learn” the upper and lower limits to the numerical scale, the increments to providing scoring, and the criteria for increasing and decreasing elements encompassed in the viral epidemiological profile 112 and/or viral prevention metric 160, etc. A machine-learning model may be used to “learn” which elements of viral biomarkers 108 have what effect on viral epidemiological profile 112, and which elements of viral epidemiological profile 112 are affected by particular nutrition elements 120 and the magnitude of effect, etc. The magnitude of the effect may be enumerated and provided as part of system 100, where nutrition elements 120 are communicated to user for their viral infection preventative properties.

Still referring to FIG. 3, machine-learning algorithms may include at least a supervised machine-learning process 328. At least a supervised machine-learning process 328, as defined herein, include algorithms that receive a training set relating a number of inputs to a number of outputs, and seek to find one or more mathematical relations relating inputs to outputs, where each of the one or more mathematical relations is optimal according to some criterion specified to the algorithm using some scoring function. For instance, a supervised learning algorithm may include a viral epidemiological profile 112 (potentially classified into viral infection categories 124), as described above as inputs, nutrient element 120 outputs, and a ranking function representing a desired form of relationship to be detected between inputs and outputs; ranking function may, for instance, seek to maximize the probability that a given input (such as nutrient amounts 144) and/or combination of inputs is associated with a given output (alleviation program 152 that incorporate nutrient elements 120 to achieve nutrient amounts 144 that are ‘best’ for viral infection category 124) to minimize the probability that a given input is not associated with a given output, for instance finding the most suitable times to consume meals, and what the meals should be, etc. Ranking function may be expressed as a risk function representing an “expected loss” of an algorithm relating inputs to outputs, where loss is computed as an error function representing a degree to which a prediction generated by the relation is incorrect when compared to a given input-output pair provided in training data 304. Persons skilled in the art, upon reviewing the entirety of this disclosure, will be aware of various possible variations of at least a supervised machine-learning process 328 that may be used to determine relation between inputs and outputs. Supervised machine-learning processes may include classification algorithms as defined above.

Further referring to FIG. 3, machine learning processes may include at least an unsupervised machine-learning process 332. An unsupervised machine-learning process 332, as used herein, is a process that derives inferences in datasets without regard to labels; as a result, an unsupervised machine-learning process 332 may be free to discover any structure, relationship, and/or correlation provided in the data. Unsupervised processes may not require a response variable; unsupervised processes may be used to find interesting patterns and/or inferences between variables, to determine a degree of correlation between two or more variables, or the like.

Still referring to FIG. 3, machine-learning module 300 may be designed and configured to create a machine-learning model 324 using techniques for development of linear regression models. Linear regression models may include ordinary least squares regression, which aims to minimize the square of the difference between predicted outcomes and actual outcomes according to an appropriate norm for measuring such a difference (e.g. a vector-space distance norm); coefficients of the resulting linear equation may be modified to improve minimization. Linear regression models may include ridge regression methods, where the function to be minimized includes the least-squares function plus term multiplying the square of each coefficient by a scalar amount to penalize large coefficients. Linear regression models may include least absolute shrinkage and selection operator (LASSO) models, in which ridge regression is combined with multiplying the least-squares term by a factor of 1 divided by double the number of samples. Linear regression models may include a multi-task lasso model wherein the norm applied in the least-squares term of the lasso model is the Frobenius norm amounting to the square root of the sum of squares of all terms. Linear regression models may include the elastic net model, a multi-task elastic net model, a least angle regression model, a LARS lasso model, an orthogonal matching pursuit model, a Bayesian regression model, a logistic regression model, a stochastic gradient descent model, a perceptron model, a passive aggressive algorithm, a robustness regression model, a Huber regression model, or any other suitable model that may occur to persons skilled in the art upon reviewing the entirety of this disclosure. Linear regression models may be generalized in an embodiment to polynomial regression models, whereby a polynomial equation (e.g. a quadratic, cubic or higher-order equation) providing a best predicted output/actual output fit is sought; similar methods to those described above may be applied to minimize error functions, as will be apparent to persons skilled in the art upon reviewing the entirety of this disclosure.

Continuing to refer to FIG. 3, machine-learning algorithms may include, without limitation, linear discriminant analysis. Machine-learning algorithm may include quadratic discriminate analysis. Machine-learning algorithms may include kernel ridge regression. Machine-learning algorithms may include support vector machines, including without limitation support vector classification-based regression processes. Machine-learning algorithms may include stochastic gradient descent algorithms, including classification and regression algorithms based on stochastic gradient descent. Machine-learning algorithms may include nearest neighbors algorithms. Machine-learning algorithms may include Gaussian processes such as Gaussian Process Regression. Machine-learning algorithms may include cross-decomposition algorithms, including partial least squares and/or canonical correlation analysis. Machine-learning algorithms may include naïve Bayes methods. Machine-learning algorithms may include algorithms based on decision trees, such as decision tree classification or regression algorithms. Machine-learning algorithms may include ensemble methods such as bagging meta-estimator, forest of randomized tress, AdaBoost, gradient tree boosting, and/or voting classifier methods. Machine-learning algorithms may include neural net algorithms, including convolutional neural net processes.

Still referring to FIG. 3, models may be generated using alternative or additional artificial intelligence methods, including without limitation by creating an artificial neural network, such as a convolutional neural network comprising an input layer of nodes, one or more intermediate layers, and an output layer of nodes. Connections between nodes may be created via the process of “training” the network, in which elements from a training data 304 set are applied to the input nodes, a suitable training algorithm (such as Levenberg-Marquardt, conjugate gradient, simulated annealing, or other algorithms) is then used to adjust the connections and weights between nodes in adjacent layers of the neural network to produce the desired values at the output nodes. This process is sometimes referred to as deep learning. This network may be trained using training data 204.

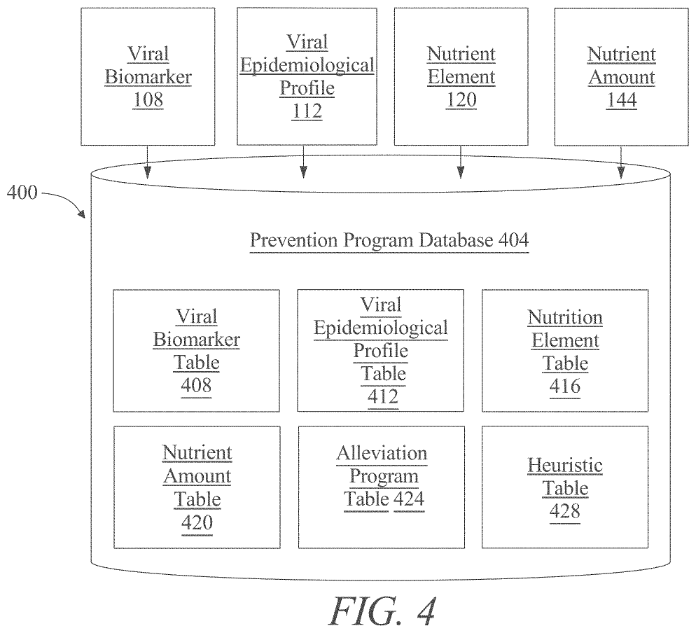

Referring now to FIG. 4, a non-limiting exemplary embodiment 400 of an alleviation program database 404 is illustrated. Viral biomarker(s) 108 for a plurality of subjects, for instance for generating a training data classifier 316, may be stored and/or retrieved in alleviation program database 404. Viral biomarker 108 data from a plurality of subjects for generating training data 304 may also be stored and/or retrieved from an alleviation program database 404. Training data for spread model 140, such as a plurality of epidemiological factors, including data from municipal health authorities, viral incidence tracking, contact tracing data, and the like, may be stored and/or retrieved in alleviation program database 404. Computing device 104 may receive, store, and/or retrieve training data 304, wearable device data, physiological sensor data, biological extraction data, and the like, from alleviation program database 404. Computing device 104 may store and/or retrieve viral classifier 128, spread model 140, nutrition machine-learning model 148, among other determinations, I/O data, models, and the like, from alleviation program database 404.

Continuing in reference to FIG. 4, alleviation program database 404 may be implemented, without limitation, as a relational database, a key-value retrieval database such as a NOSQL database, or any other format or structure for use as a database that a person skilled in the art would recognize as suitable upon review of the entirety of this disclosure. Alleviation program database 404 may alternatively or additionally be implemented using a distributed data storage protocol and/or data structure, such as a distributed hash table and the like. Alleviation program database 404 may include a plurality of data entries and/or records, as described above. Data entries in an alleviation program database 404 may be flagged with or linked to one or more additional elements of information, which may be reflected in data entry cells and/or in linked tables such as tables related by one or more indices in a relational database. Persons skilled in the art, upon reviewing the entirety of this disclosure, will be aware of various ways in which data entries in a database may store, retrieve, organize, and/or reflect data and/or records as used herein, as well as categories and/or populations of data consistent with this disclosure.

Further referring to FIG. 4, alleviation program database 404 may include, without limitation, viral biomarker table 408, viral epidemiological profile table 412, nutrition element table 416, nutrient amount table 420, alleviation program table 424, and/or heuristic table 428. Determinations by a machine-learning process, machine-learning model, ranking function, and/or classifier, may also be stored and/or retrieved from the alleviation program database 404. As a non-limiting example, alleviation program database 404 may organize data according to one or more instruction tables. One or more alleviation program database 404 tables may be linked to one another by, for instance in a non-limiting example, common column values. For instance, a common column between two tables of alleviation program database 404 may include an identifier of a submission, such as a form entry, textual submission, accessory device tokens, local access addresses, metrics, and the like, for instance as defined herein; as a result, a search by a computing device 104 may be able to retrieve all rows from any table pertaining to a given submission or set thereof. Other columns may include any other category usable for organization or subdivision of data, including types of data, names and/or identifiers of individuals submitting the data, times of submission, and the like; persons skilled in the art, upon reviewing the entirety of this disclosure, will be aware of various ways in which data from one or more tables may be linked and/or related to data in one or more other tables.

Continuing in reference to FIG. 4, in a non-limiting embodiment, one or more tables of an alleviation program database 404 may include, as a non-limiting example, a viral biomarker table 408, which may include categorized identifying data, as described above, including genetic data, epigenetic data, viral markers of infection, physiological data, biological extraction data, and the like. Viral biomarker table 408 may include viral biomarker 108 categories according to gene expression patterns, SNPs, mutations, cytokine concentrations, protein phosphorylation data, tissue stress data, data concerning metabolism of nutrition elements 120, such as user-specific pharmacokinetics, nutrient absorption, etc.; categories may include tables linked to mathematical expressions that describe the impact and/por relationship of each viral biomarker 108 datum on viral epidemiological profile 112, for instance threshold values for the cytokine expression biomarkers, etc., as it relates to viral infection, viral infection category 124, etc. One or more tables may include viral epidemiological profile table 412, which may include data regarding viral biomarker 108, thresholds, scores, metrics, values, categorizations, and the like, that system 100 may use to calculate, derive, filter, retrieve and/or store current viral infection levels, viral infection types, likelihood of currently having an infection, probability of future infection, nutritional deficiency, and the like. One or more tables may include nutrition element table 416, which may include data on nutrition elements 120 such as foods, ingredients, supplements, bioactive ingredients, phytonutrients, antioxidants, pharmaceuticals, and the like, for instance as classified to viral infection category 124, classified to data from alike subjects with similar viral biomarker(s) 108, as related to viral epidemiological profile 112, and the like, that system 100 may use to calculate, derive, filter, retrieve and/or store nutrition elements 120. One or more tables may include nutrient amount table 420, which may include functions, model, equations, algorithms, and the like, using to calculate or derive nutrient amounts 144 relating to viral epidemiological profile 112 and/or viral infection category 124, may include nutrient amounts 144 organized by nutrient, nutrient classification, user age, sex, viral infection severity, infection risk, climate, time of year, etc. One of more tables may include an alleviation program table 424, which may include nutrition element 120 identifiers, serving sizes, and/or timetables associated with nutrition elements 120, regarding times to eat, identifiers of meals, recipes, ingredients, schedules, diet types, and the like. One or more tables may include, without limitation, a heuristic table 428, which may organize rankings, scores (e.g. viral prevention metric) models (e.g. spread model 140), classifiers (e.g. viral classifier 128), outcomes, functions, numerical values, scales, arrays, matrices, and the like, that represent determinations, probabilities, metrics, parameters, values, and the like, include one or more inputs describing potential mathematical relationships, as described herein.

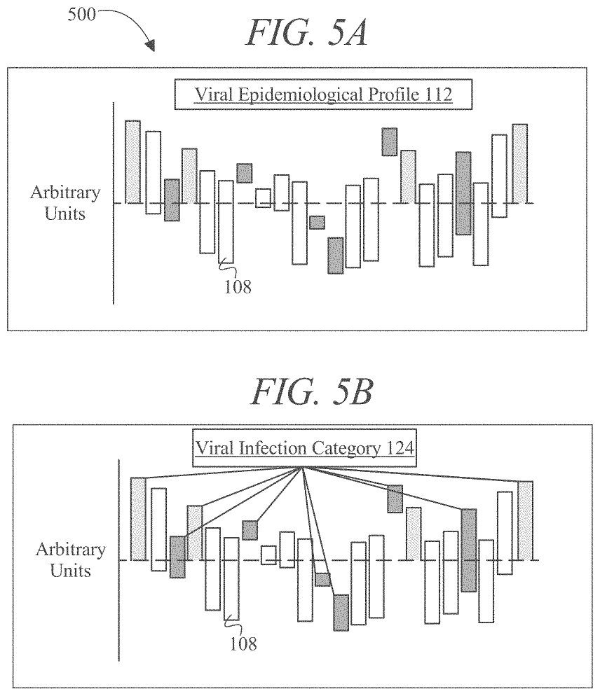

Referring now to FIGS. 5A and 5B, a non-limiting exemplary embodiment 500 of a viral epidemiological profile 112 is illustrated. Viral epidemiological profile 112 may include a variety of viral biomarker 108 categories, for instance 22 distinct categories, as shown in FIGS. 5A and 5B. each viral biomarker 108 may be assigned a value, such as an arbitrary value, where some viral biomarkers 108, such as those shaded in light grey, may relate to absolute scales from [0, x], where x is a maximal value and the range of values for the viral biomarker 108 cannot be below a ‘zero amount’. Some viral biomarkers 108, such as those shaded in dark grey, may relate to cytokine levels, wherein, the viral biomarker 108 is enumerated as a ‘box plot’ that illustrates the range of expression in a population of users organized according to, for instance tissue type, normal vs infected state, etc.). In such an example, the dashed line may relate to a ‘normal threshold’ above which is elevated gene expression, below which is decreased expression level. Each viral biomarker 108 may have associated with it a numerical score, or some other identifying mathematical value that computing device 104 may assign. Persons skilled in the art may appreciate that for each user, any number of viral biomarkers 108 may be enumerated and assigned a value according to viral epidemiological profile machine-learning model 116. Viral epidemiological profile 112 may be graphed, or otherwise displayed, according to the enumeration by viral epidemiological profile machine-learning model 116. Each bar of the bar graph, or combinations of bar graph categories, may instruct a classification of a user's viral epidemiological profile 112 to a viral infection category 124.

Still referring now to FIGS. 5A and 5B, in non-limiting exemplary illustrations viral epidemiological profile 112 may be classified to a viral infection category 124. Some and/or all of the viral biomarkers 108 summarized in viral epidemiological profile 112 may be used to classify an individual to a particular viral infection category 124. For instance, as shown in FIG. 5B, ten of the 22 viral biomarker 108 categories may be used to classify viral epidemiological profile 112 to one or more viral infection categories 124. Viral epidemiological profile machine-learning model 116 may be trained to assign viral biomarker 108 to a viral infection category 124, wherein computing device 104 may know the identity of viral infection category 124 according to which viral infection category 124 has the most identifying data points. Alternatively or additionally, viral epidemiological profile 112 may include categorizations of epidemiological factors, as described above, enumerated according to viral epidemiological profile machine-learning model 116 assigned values. Such data categories may work to increase accuracy of viral infection category 124 by thorough consideration of viral outbreak factors.

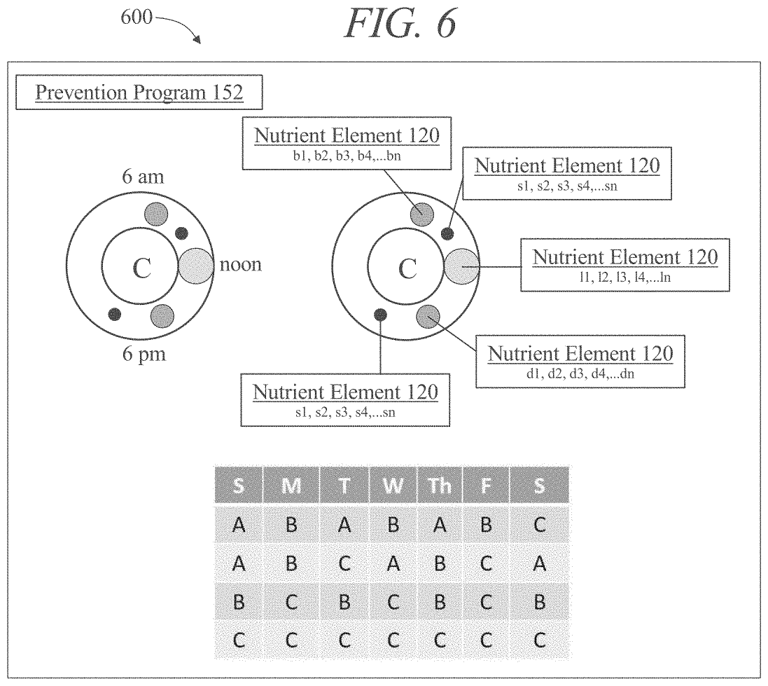

Referring now to FIG. 6, a non-limiting exemplary embodiment 600 of a viral alleviation program 152 is illustrated. Alleviation program 152 may include a schedule for arranging nutrition elements 120, according to for instance a 24-hour timetable, as designated on the left, where consumption is planned along a user's typical day-night cycle, beginning at ˜6 am until just after 6 pm. Nutrition element 120 may include breakfast (denoted as mid-sized dark grey circle), which may correspond to a file of breakfast-related plurality of nutrition elements 120 (denoted b1, b2, b3, b4 . . . bn, to the nth breakfast item). Nutrition element 120 may include snacks eaten throughout the day to, for instance achieve nutrient amounts 144 missing from meals (denoted as small black circles), which may correspond to a file of snacking-related plurality of nutrition elements 120 (denoted s1, s2, s3, s4 . . . sn, to the nth snacking item). Nutrition element 120 may include dinner (denoted as large-sized light grey circle), which may correspond to a file of dinner-related plurality of nutrition elements 120 (denoted d1, d2, d3, d4 . . . dn, to the nth dinner item). Alleviation program 152 may include a variety of diets, as denoted in the monthly schedule at the bottom, Sunday through Saturday. Alleviation program 152 ‘C’ is shown, which may be an idealistic goal for user to achieve by the end of the month, where alleviation program ‘A’ and ‘B’ are intermediate plans intended to wean user to the ‘ideal’ plan. Nutrition elements 120 classified by ‘meal type’ may be further modified by ‘A’ and ‘B’ according to user preferences 148 collected by computing device 104 throughout the process. Circle sizes, denoting nutrition element 120 classes may relate to portion sizes, which are graphed along the circle corresponding to the times they are expected to be consumed. User may select which nutrition element 120 from each category is to be consumed, and when it was consumed, to arrive at viral prevention metric 160; this is to say viral prevention metric 160 may be iteratively updated as a function of the user-selected nutrition element 120 output from system 100.

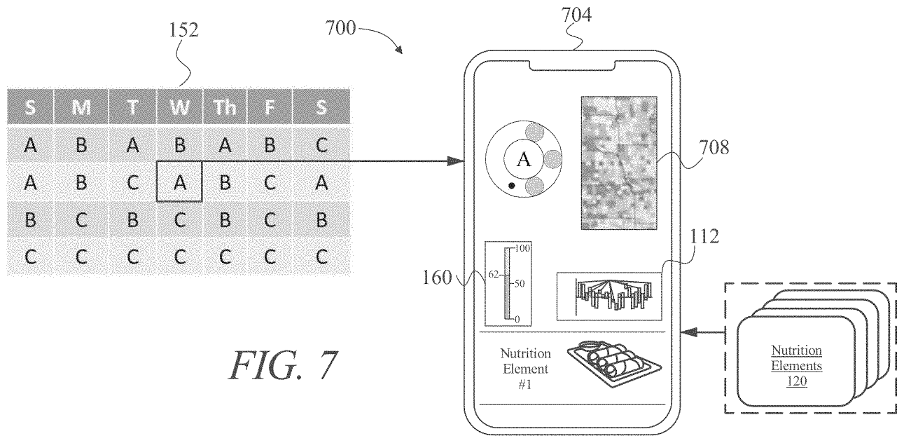

Referring now to FIG. 7, a non-limiting exemplary embodiment 700 of a user device 704 is illustrated. User device 704 may include computing device 104, a “smartphone,” cellular mobile phone, desktop computer, laptop, tablet computer, internet-of-things (IOT) device, wearable device, among other devices. User device 704 may include any device that is capable for communicating with computing device 104, alleviation program database 404, or able to receive, transmit, and/or display, via a graphical user interface, viral epidemiological profile 112, nutrition element 120, alleviation program 152, viral prevention metric 160, among other outputs from system 100. User device 704 may provide a viral epidemiological profile 112, for instance as a collection of metrics determined from viral biomarker 108 data. User device 704 may provide viral infection category 124 that was determined as a function of parameters/determinations enumerated in viral epidemiological profile 112. User device 704 may provide data concerning nutrient amounts 144, including the levels of specific nutrients, nutrient ranges, nutrients to avoid, etc. User device 704 may link timing of foods to preemptive ordering interface for ordering a nutrition element 120, for instance and without limitation, through a designated mobile application, mapping tool or application, etc., and a radial search method about a user's current location as described in U.S. Nonprovisional application Ser. No. 17/087,745, filed Nov. 3, 2020, titled “A METHOD FOR AND SYSTEM FOR PREDICTING ALIMENTARY ELEMENT ORDERING BASED ON BIOLOGICAL EXTRACTION,” the entirety of which is incorporated herein by reference. User device 704 may display nutrient elements 120 as a function of location, for instance and without limitation, as described in User device 704 may link nourishment consumption program 120 to a scheduling application, such as a ‘calendar’ feature on user device, which may set audio-visual notifications, timers, alarms, and the like. May select locations for nutrition elements 120 based on entity affinity to viral infection research, viral infection charity, etc.

Continuing in reference to FIG. 7, user device 704 may provide viral epidemiology map 708. “Viral epidemiology map,” as used in this disclosure, is a graphical display of viral epidemiology factors as summarized in viral epidemiology profile 112. Viral epidemiology map 708 may include a heat map, or coloring scale of geographical regions on a map as a function of viral risk, viral case numbers, etc. Viral epidemiology map 708 may include any graphical, text-based, icon-based, or the like, display of geographic information software (GIS) meant to convey the phylogenetics and epidemiology of a virus in a human population as generated from spread model 140 using viral spread training data 136. Viral epidemiology map 708 map include applying spread model 140 outputs to a geographical mapping about the user, for instance using the Internet and a mapping tool, algorithm, or mapping application (e.g. GOOGLE MAPS), or the like. User device 704 may determine a user's current location and apply the outputs of spread model 140 to illustrate the current viral epidemiological landscape. User device 704 may update viral infection profile 112 as a function of the viral epidemiology map 708. User device 704 may update viral prevention metric 160 as a function of user participation in alleviation program 152, as indicated from selected nutrition elements 120.

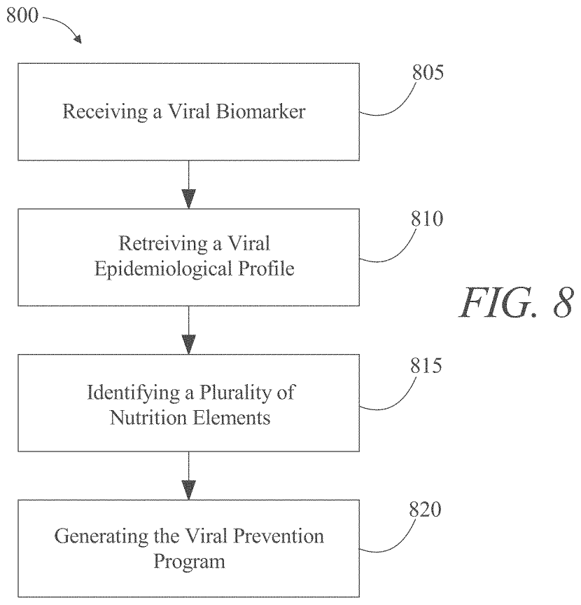

Referring now to FIG. 8, an exemplary embodiment 800 of a method for generating a viral alleviation program is illustrated. At step 805, the method including receiving, by a computing device 104, at least a viral biomarker 108 relating to a user. Receiving at least a viral biomarker 108 may include receiving a result of one or more tests relating the user; this may be implemented, without limitation, as described above in FIGS. 1-7.

Still referring to FIG. 8, at step 810, method includes retrieving, by the computing device 104, a viral epidemiological profile 112 related to the user. of a plurality of epidemiological factors as a function of at least a viral biomarker 108. Retrieving the viral epidemiological profile 112 may include receiving viral epidemiological profile 112 training data, training a viral epidemiological profile machine-learning model 116 with training data that includes a plurality of data entries wherein each entry correlates viral biomarkers 108 to a plurality of epidemiological factors, and generating the viral epidemiological profile 112 as a function of the viral epidemiological profile machine-learning model 116 and at least a viral biomarker 108; this may be implemented, without limitation, as described above in FIGS. 1-7.

Continuing in reference to FIG. 8, at step 815, method includes identifying, by the computing device 104 and using the viral epidemiological profile 112, a plurality of nutrition elements 120 for the user, wherein identifying includes assigning the viral epidemiological profile 112 to a viral infection category 124, wherein the viral infection category 124 is a determination about a current viral epidemiological state of the user, calculating, according to the viral infection category 124, a plurality of nutrient amounts 144, wherein calculating a plurality of nutrient amounts 144 includes determining an effect of the plurality of nutrient amounts 144 on the viral epidemiological profile 112, and calculating the plurality of nutrient amounts 144 as a function of the effect, wherein the plurality of nutrient amounts 144 is a plurality of amounts intended to result in prevention of viral infection. Identifying, as a function of the plurality of nutrient amounts 144, the plurality of nutrition elements 120, wherein the plurality of nutrition elements 132 are intended to prevent viral infection as a function of the viral infection category 124. Assigning the viral epidemiological profile 112 to a viral infection category 124 may include training a viral classifier 128 using a viral classification machine-learning process 132 and training data which includes a plurality of data entries wherein each data entry correlates viral biomarkers 108 to a viral infection category 124 and assigning the viral infection category 124 as a function of the viral classifier and the viral epidemiological profile 112. Determining the effect of the plurality of nutrient amounts 144 on the viral epidemiological profile 112 may include receiving viral spread training data 136, generating a spread model 140, wherein the spread model 140 is a machine-learning model trained with the viral spread training data 136 which includes a plurality of data entries that models a plurality of effects of the plurality of nutrient amounts 144 on viral spread rates, and determining the effect of the plurality of nutrient amounts 144 as a function of the spread model 140. Calculating the plurality of nutrient amounts 144 may include determining the plurality of nutrient amounts 144 as a function of at least a viral biomarker 108 and the plurality of effects. Calculating nutrient amounts 144 may include generating training data using the plurality of effects of the plurality of nutrient amounts 144 identified according to the viral infection category 124, training a nutrition machine-learning model 148 according to the training data, wherein training data includes a plurality of data entries that correlates the magnitude of nutrient effect to a plurality of nutrient amounts for each viral infection category 124, and calculating nutrient amounts 144 as a function of the nutrition machine learning model 148 and the viral infection category 124; this may be implemented, without limitation, as described above in FIGS. 1-8.

Continuing in reference to FIG. 8, at step 820, method includes generating, by the computing device 104, using the plurality of nutrition elements 120, the viral alleviation program 152. Generating viral alleviation program 152 may include generating an alleviation program classifier 156 using a nourishment classification machine-learning process to classify the plurality of nutrient amounts 144 to the plurality of nutrition elements 120 and outputting the plurality of nutrition elements 120 as a function of the alleviation program classifier 156. Generating viral alleviation program 152 may include generating a viral prevention metric 160, wherein the viral prevention metric 160 reflects the level of user participation in viral alleviation program 152. Generating viral alleviation program 152 may include calculating a change in incidence of viral infection as a function of adhering to alleviation program 152.; this may be implemented, without limitation, as described above in FIGS. 1-7.

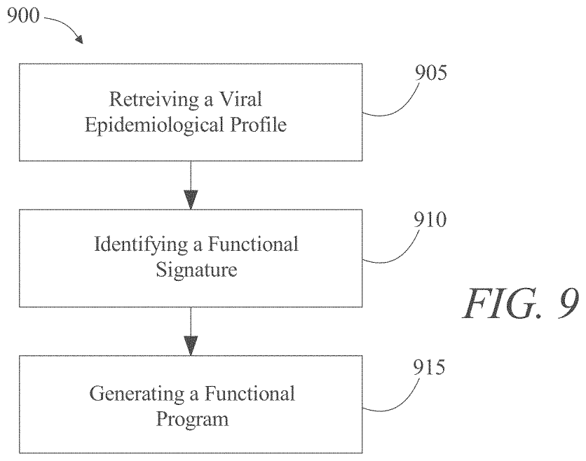

Now referring to FIG. 9, an exemplary embodiment 900 of a method for generating an integrative program is illustrated. At step 905, the method including retrieving, by a computing device 104, a viral epidemiological profile 112 related to a user. Retrieving the viral epidemiological profile 112 may include receiving viral epidemiological profile 112 training data, training a viral epidemiological profile machine-learning model 116 with training data that includes a plurality of data entries wherein each entry correlates viral biomarkers 108 to a plurality of epidemiological factors, and generating the viral epidemiological profile 112 as a function of the viral epidemiological profile machine-learning model 116 and at least a viral biomarker 108; this may be implemented, without limitation, as described above in FIGS. 1-8.

Still referring to FIG. 9, at step 910, computing device 104 identifies integrative signature 204. Computing device 104 identifies integrative signature 204 as a function of receiving a behavioral indicator 208. Computing device 104 identifies integrative signature 204 as a function of behavioral indicator 208 and viral epidemiologic disorder using an integrative machine-learning model 212. This may be implemented, without limitation, as described above in FIGS. 1-8.

[symbol]

Still referring to FIG. 9, at step, 915, computing device 104 generates an integrative program 616 as a function of integrative signature 204. This may be implemented, without limitation, as described above in FIGS. 1-8.

It is to be noted that any one or more of the aspects and embodiments described herein may be conveniently implemented using one or more machines (e.g., one or more computing devices that are utilized as a user computing device for an electronic document, one or more server devices, such as a document server, etc.) programmed according to the teachings of the present specification, as will be apparent to those of ordinary skill in the computer art. Appropriate software coding may readily be prepared by skilled programmers based on the teachings of the present disclosure, as will be apparent to those of ordinary skill in the software art. Aspects and implementations discussed above employing software and/or software modules may also include appropriate hardware for assisting in the implementation of the machine executable instructions of the software and/or software module.

Such software may be a computer program product that employs a machine-readable storage medium. A machine-readable storage medium may be any medium that is capable of storing and/or encoding a sequence of instructions for execution by a machine (e.g., a computing device) and that causes the machine to perform any one of the methodologies and/or embodiments described herein. Examples of a machine-readable storage medium include, but are not limited to, a magnetic disk, an optical disc (e.g., CD, CD-R, DVD, DVD-R, etc.), a magneto-optical disk, a read-only memory “ROM” device, a random access memory “RAM” device, a magnetic card, an optical card, a solid-state memory device, an EPROM, an EEPROM, and any combinations thereof. A machine-readable medium, as used herein, is intended to include a single medium as well as a collection of physically separate media, such as, for example, a collection of compact discs or one or more hard disk drives in combination with a computer memory. As used herein, a machine-readable storage medium does not include transitory forms of signal transmission.

Such software may also include information (e.g., data) carried as a data signal on a data carrier, such as a carrier wave. For example, machine-executable information may be included as a data-carrying signal embodied in a data carrier in which the signal encodes a sequence of instruction, or portion thereof, for execution by a machine (e.g., a computing device) and any related information (e.g., data structures and data) that causes the machine to perform any one of the methodologies and/or embodiments described herein.

Examples of a computing device include, but are not limited to, an electronic book reading device, a computer workstation, a terminal computer, a server computer, a handheld device (e.g., a tablet computer, a smartphone, etc.), a web appliance, a network router, a network switch, a network bridge, any machine capable of executing a sequence of instructions that specify an action to be taken by that machine, and any combinations thereof. In one example, a computing device may include and/or be included in a kiosk.

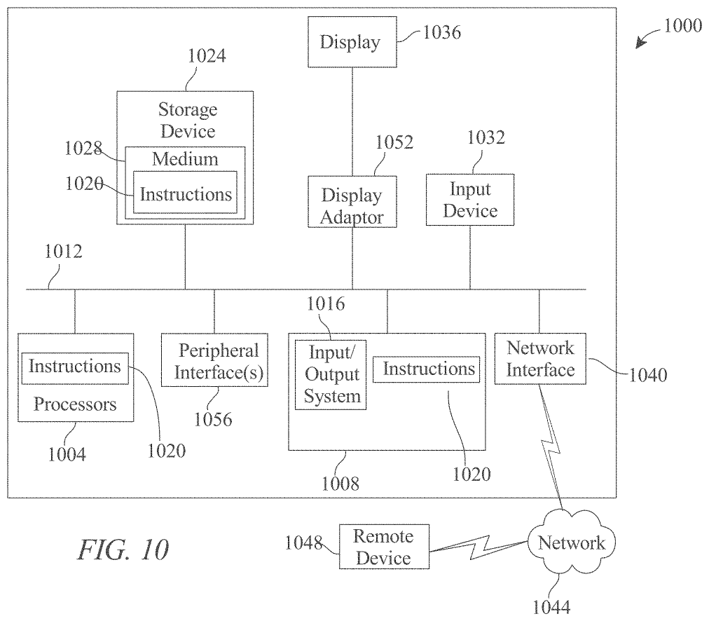

FIG. 10 shows a diagrammatic representation of one embodiment of a computing device in the exemplary form of a computer system 1000 within which a set of instructions for causing a control system to perform any one or more of the aspects and/or methodologies of the present disclosure may be executed. It is also contemplated that multiple computing devices may be utilized to implement a specially configured set of instructions for causing one or more of the devices to perform any one or more of the aspects and/or methodologies of the present disclosure. Computer system 1000 includes a processor 1004 and a memory 1008 that communicate with each other, and with other components, via a bus 1012. Bus 1012 may include any of several types of bus structures including, but not limited to, a memory bus, a memory controller, a peripheral bus, a local bus, and any combinations thereof, using any of a variety of bus architectures.

Processor 1004 may include any suitable processor, such as without limitation a processor incorporating logical circuitry for performing arithmetic and logical operations, such as an arithmetic and logic unit (ALU), which may be regulated with a state machine and directed by operational inputs from memory and/or sensors; processor 1004 may be organized according to Von Neumann and/or Harvard architecture as a non-limiting example. Processor 1004 may include, incorporate, and/or be incorporated in, without limitation, a microcontroller, microprocessor, digital signal processor (DSP), Field Programmable Gate Array (FPGA), Complex Programmable Logic Device (CPLD), Graphical Processing Unit (GPU), general purpose GPU, Tensor Processing Unit (TPU), analog or mixed signal processor, Trusted Platform Module (TPM), a floating point unit (FPU), and/or system on a chip (SoC).

Memory 1008 may include various components (e.g., machine-readable media) including, but not limited to, a random-access memory component, a read only component, and any combinations thereof. In one example, a basic input/output system 1016 (BIOS), including basic routines that help to transfer information between elements within computer system 1000, such as during start-up, may be stored in memory 1008. Memory 1008 may also include (e.g., stored on one or more machine-readable media) instructions (e.g., software) 1020 embodying any one or more of the aspects and/or methodologies of the present disclosure. In another example, memory 1008 may further include any number of program modules including, but not limited to, an operating system, one or more application programs, other program modules, program data, and any combinations thereof.

Computer system 1000 may also include a storage device 1024. Examples of a storage device (e.g., storage device 1024) include, but are not limited to, a hard disk drive, a magnetic disk drive, an optical disc drive in combination with an optical medium, a solid-state memory device, and any combinations thereof. Storage device 1024 may be connected to bus 1012 by an appropriate interface (not shown). Example interfaces include, but are not limited to, SCSI, advanced technology attachment (ATA), serial ATA, universal serial bus (USB), IEEE 1394 (FIREWIRE), and any combinations thereof. In one example, storage device 1024 (or one or more components thereof) may be removably interfaced with computer system 1000 (e.g., via an external port connector (not shown)). Particularly, storage device 1024 and an associated machine-readable medium 1028 may provide nonvolatile and/or volatile storage of machine-readable instructions, data structures, program modules, and/or other data for computer system 1000. In one example, software 1020 may reside, completely or partially, within machine-readable medium 1028. In another example, software 1020 may reside, completely or partially, within processor 1004.

Computer system 1000 may also include an input device 1032. In one example, a user of computer system 1000 may enter commands and/or other information into computer system 1000 via input device 1032. Examples of an input device 1032 include, but are not limited to, an alpha-numeric input device (e.g., a keyboard), a pointing device, a joystick, a gamepad, an audio input device (e.g., a microphone, a voice response system, etc.), a cursor control device (e.g., a mouse), a touchpad, an optical scanner, a video capture device (e.g., a still camera, a video camera), a touchscreen, and any combinations thereof. Input device 1032 may be interfaced to bus 1012 via any of a variety of interfaces (not shown) including, but not limited to, a serial interface, a parallel interface, a game port, a USB interface, a FIREWIRE interface, a direct interface to bus 1012, and any combinations thereof. Input device 1032 may include a touch screen interface that may be a part of or separate from display 1036, discussed further below. Input device 1032 may be utilized as a user selection device for selecting one or more graphical representations in a graphical interface as described above.

A user may also input commands and/or other information to computer system 1000 via storage device 1024 (e.g., a removable disk drive, a flash drive, etc.) and/or network interface device 1040. A network interface device, such as network interface device 1040, may be utilized for connecting computer system 1000 to one or more of a variety of networks, such as network 1044, and one or more remote devices 1048 connected thereto. Examples of a network interface device include, but are not limited to, a network interface card (e.g., a mobile network interface card, a LAN card), a modem, and any combination thereof. Examples of a network include, but are not limited to, a wide area network (e.g., the Internet, an enterprise network), a local area network (e.g., a network associated with an office, a building, a campus or other relatively small geographic space), a telephone network, a data network associated with a telephone/voice provider (e.g., a mobile communications provider data and/or voice network), a direct connection between two computing devices, and any combinations thereof. A network, such as network 1044, may employ a wired and/or a wireless mode of communication. In general, any network topology may be used. Information (e.g., data, software 1020, etc.) may be communicated to and/or from computer system 1000 via network interface device 1040.

Computer system 1000 may further include a video display adapter 1052 for communicating a displayable image to a display device, such as display device 1036. Examples of a display device include, but are not limited to, a liquid crystal display (LCD), a cathode ray tube (CRT), a plasma display, a light emitting diode (LED) display, and any combinations thereof. Display adapter 1052 and display device 1036 may be utilized in combination with processor 1004 to provide graphical representations of aspects of the present disclosure. In addition to a display device, computer system 1000 may include one or more other peripheral output devices including, but not limited to, an audio speaker, a printer, and any combinations thereof. Such peripheral output devices may be connected to bus 1012 via a peripheral interface 1056. Examples of a peripheral interface include, but are not limited to, a serial port, a USB connection, a FIREWIRE connection, a parallel connection, and any combinations thereof.

The foregoing has been a detailed description of illustrative embodiments of the invention. Various modifications and additions may be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A system for generating a viral alleviation program, the system comprising a computing device, wherein the computing device is configured to:
produce a viral epidemiological profile related to the user;
identify an integrative signature as a function of the viral epidemiological profile, wherein
identifying further comprises:
receiving a behavioral indicator; and
identifying the integrative signature as a function of the behavioral indicator and the viral epidemiological profile using an integrative machine-learning model; and
generate an integrative program as a function of the integrative signature.

2. The system of claim 1, wherein identifying the integrative signature further comprises:
receiving an integrative training set that correlates a plurality of viral epidemiological profiles and a plurality of behavioral indicators to an integrative signature
training the integrative machine-learning model as a function of the integrative training set; and
identifying the integrative signature as a function of the integrative machine-learning model, wherein the integrative machine-learning model outputs the integrative signature as a function of the behavioral indicator and the viral epidemiological profile inputs.

3. The system of claim 1, wherein producing the viral epidemiological profile further comprises identifying a contagion element.

4. The system of claim 1, wherein identifying the integrative signature further comprises determining a travel routine.

5. The system of claim 4, wherein determining the travel routine further comprises identifying a geographical prevalence.

6. The system of claim 1, wherein identifying the integrative signature further comprises determining a susceptibility element.

7. The system of claim 6, wherein the susceptibility element includes an immune element.

8. The system of claim 6, wherein determining the susceptibility element further comprises identifying a contact element.

9. The system of claim 1, wherein generating the integrative program includes generating a viral alleviation program.

10. The system of claim 9, wherein the generating the viral alleviation program includes generating a viral prevention metric.

11. A method for generating a viral alleviation program, the method comprising:
retrieving, by the computing device, a viral epidemiological profile related to the user;
identifying, by the computing device, an integrative signature as a function of the viral epidemiological profile, wherein identifying further comprises:
receiving a behavioral indicator; and
identifying the integrative signature as a function of the behavioral indicator and the viral epidemiological profile using an integrative machine-learning model; and
generating, by the computing device, an integrative program as a function of the integrative signature.

12. The method of claim 11, wherein identifying the integrative signature further comprises:
receiving an integrative training set that correlates a plurality of viral epidemiological profiles and a plurality of behavioral indicators to an integrative signature
training the integrative machine-learning model as a function of the integrative training set; and
identifying the integrative signature as a function of the integrative machine-learning model, wherein the integrative machine-learning model outputs the integrative signature as a function of the behavioral indicator and the viral epidemiological profile inputs.

13. The method of claim 11, wherein producing the viral epidemiological profile further comprises identifying a contagion element.

14. The method of claim 11, wherein identifying the integrative signature further comprises determining a travel routine.

15. The method of claim 14, wherein determining the travel routine further comprises identifying a geographical prevalence.

16. The method of claim 11, wherein identifying the integrative signature further comprises determining a susceptibility element.

17. The method of claim 16, wherein the susceptibility element includes an immune element.

18. The method of claim 16, wherein determining the susceptibility element further comprises identifying a contact element.

19. The method of claim 11, wherein generating the integrative program includes generating a viral alleviation program.

20. The method of claim 19, wherein the generating the viral alleviation program includes generating a viral prevention metric.
